SEQUENCE LISTING (28 sequences)

SEQ ID NO: 1 - DNA, 40 bp, Artificial Sequence
  Description of Artificial Sequence Synthetic primer
catatgaattcaccatgagaaaacatgtactagctgcatc

SEQ ID NO: 2 - DNA, 43 bp, Artificial Sequence
  Description of Artificial Sequence Synthetic primer
gatatgcggccgctctagatcaggaattgcattcctcgcagtg

SEQ ID NO: 3 - DNA, 34 bp, Artificial Sequence
  Description of Artificial Sequence Synthetic primer
ggacgaattcaccatggggacacaagccctgcag

SEQ ID NO: 4 - DNA, 32 bp, Artificial Sequence
  Description of Artificial Sequence Synthetic primer
ctcgctcatctccgctctctgaggatcccagg

SEQ ID NO: 5 - DNA, 39 bp, Artificial Sequence
  Description of Artificial Sequence Synthetic oligonucleotide
caatagtcgacgaattcaccatggctctgggcgcagcgg

SEQ ID NO: 6 - DNA, 42 bp, Artificial Sequence
  Description of Artificial Sequence Synthetic oligonucleotide
gtattgcggccgctctagattagtgtctctgacaagtgtgaa

SEQ ID NO: 7 - DNA, 32 bp, Artificial Sequence
  Description of Artificial Sequence Synthetic oligonucleotide
ctaacggatccaccatggccgcgttgatgcgg

SEQ ID NO: 8 - DNA, 33 bp, Artificial Sequence
  Description of Artificial Sequence Synthetic oligonucleotide
gattcgaattctcaaattttctgacacacatgg

SEQ ID NO: 9 - DNA, 31 bp, Artificial Sequence
  Description of Artificial Sequence Synthetic primer
gggaattcgcgacgatgagggcgcggccgca

SEQ ID NO: 10 - DNA, 33 bp, Artificial Sequence
  Description of Artificial Sequence Synthetic primer
gggcggccgcagggcctcacttgcagacatagc

SEQ ID NO: 11 - DNA, 31 bp, Artificial Sequence
  Description of Artificial Sequence Synthetic primer
ctaacggatccaccatgctggaggagccccg

SEQ ID NO: 12 - DNA, 31 bp, Artificial Sequence
  Description of Artificial Sequence Synthetic primer
gattcgaattctcacttacacacattcaccc

SEQ ID NO: 13 - DNA, 31 bp, Artificial Sequence
  Description of Artificial Sequence Synthetic primer
ctaacggatccaccatgggcagcgcccaccc

SEQ ID NO: 14 - DNA, 30 bp, Artificial Sequence
  Description of Artificial Sequence Synthetic primer
gattcgaattctcacttgcagacgctgacc

SEQ ID NO: 15 - DNA, 32 bp, Artificial Sequence
  Description of Artificial Sequence Synthetic primer
ctaacggatccaccatggaaaggcacccaccc

SEQ ID NO: 16 - DNA, 32 bp, Artificial Sequence
  Description of Artificial Sequence Synthetic primer
gattcgaattcttacttgcaagtgtggacatc

SEQ ID NO: 17 - DNA, 32 bp, Artificial Sequence
  Description of Artificial Sequence Synthetic primer
ctaacggatccaccatggacagggcggcgctc

SEQ ID NO: 18 - DNA, 32 bp, Artificial Sequence; identical to SEQ ID NO: 16
  Description of Artificial Sequence Synthetic primer

SEQ ID NO: 19 - DNA, 34 bp, Artificial Sequence
  Description of Artificial Sequence Synthetic primer
ctaacggatccaccatgggggaacctgtttatgc

SEQ ID NO: 20 - DNA, 31 bp, Artificial Sequence
  Description of Artificial Sequence Synthetic primer
gattctctagactagatatagaccccaaacc

SEQ ID NO: 21 - DNA, 38 bp, Artificial Sequence
  Description of Artificial Sequence Synthetic primer
ctaacggatccaccatgtttctttcaaagccttctgtg

SEQ ID NO: 22 - DNA, 33 bp, Artificial Sequence
  Description of Artificial Sequence Synthetic primer
gattcgaattcttagggttttctcccgggtttg

SEQ ID NO: 23 - DNA, 32 bp, Artificial Sequence
  Description of Artificial Sequence Synthetic primer
ctaacgattcaacatgctgagaccgggtggtg

SEQ ID NO: 24 - DNA, 30 bp, Artificial Sequence
  Description of Artificial Sequence Synthetic primer
gattctctagatcaggtctggtccagccac

SEQ ID NO: 25 - DNA, 30 bp, Artificial Sequence
  Description of Artificial Sequence Synthetic primer
ctaactctagaatgttggatggccttggag

SEQ ID NO: 26 - DNA, 30 bp, Artificial Sequence; identical to SEQ ID NO: 24
  Description of Artificial Sequence Synthetic primer

SEQ ID NO: 27 - DNA, 55 bp, Artificial Sequence
  Description of Artificial Sequence Synthetic oligonucleotide
ctagcgagaacaaaggagattcaaaggagatcaaaggagatcaaaggactagttc

SEQ ID NO: 28 - DNA, 55 bp, Artificial Sequence
  Description of Artificial Sequence Synthetic oligonucleotide
gctcttgtttcctctaagtttcctctagtttcctctagtttcctgatcaagagct

(19) United States
(12) Patent Application Publication
(10) Pub. No.: US 2007/0196872 A1
(43) Pub. Date: Aug. 23, 2007

(54) Materials and methods for identifying agents that modulate Norrin, Norrin mimetics, and agents identified thereby

(75) Inventors: Frederick Bex, Newtown Square, PA (US); Bheem M. Bhat, West Chester, PA (US)

Correspondence Address: DRINKER BIDDLE & REATH (DC), 1500 K STREET, N.W., SUITE 1100, WASHINGTON, DC, 20005-1209, US

(73) Assignee: Wyeth, Madison, NJ (US)

(21) Appl. No.: 11/702,613

(22) Filed: Feb. 6, 2007

Related U.S. Application Data
(60) Provisional application No. 60/765,760, filed on Feb. 7, 2006.

(51) Int. Cl. G01N 33/567; C12N 5/06

(52) U.S. Cl. 435/7.2; 435/325

(57) ABSTRACT

The specification discloses materials and methods for screening and identifying reagents, which modulate Norrin activity as it relates to Wnt pathway signaling. Preferably, agents identified thereby modulate bone remodeling and/or lipid levels, and can be Norrin mimetics and Norrin agonists, as well as other agonists and mimetics of the LRP5/Norrin/Frizzled4 complex.

### CROSS REFERENCE TO RELATED APPLICATIONS

This application claims benefit of U.S. Provisional Application No. 60/765,760 filed Feb. 7, 2006, which is herein incorporated by reference in its entirety.

### REFERENCE TO A SEQUENCE LISTING

The sequence listing submitted herewith containing SEQ ID NOS: 1-28 are incorporated herein by reference for all purposes.

### FIELD OF THE INVENTION

The present invention relates to materials and methods of regulating the Norrin gene or the Norrin protein, Norrin mimetics, or agents that interact with Norrin and thereby modulate its activity in the LRP5/Norrin/Frizzled4 complex.

### BACKGROUND

Mutations in Norrin or Norrie disease protein (NDP or Ndph) leads to Norrie disease (ND), an X-linked recessive neurological syndrome (Berger et al., 1992 Nat. Genet. 1:199-203; and Chen et al., 1992 Nat. Genet. 1: 204-208; for example, NCBI Accession Nos. AAH29901, BC029901, and CAA46639 for human sequences, and CAA58725, CAA63134, and X83794 for Mus musculus sequences). The gene encoding NDP is located at Xp11.4 on the human genome. The disease characteristics include retinal dysplasia, blindness, and mental retardation. NDP knockout mice have an eye phenotype, which resembles human Norrie disease (Rhem et al., 2002 J Neuroscience 22: 4286-4292) and show failure in retinal angiogenesis. The gene is also tied to Coats disease (retinal telangiectasis), X-linked exudative vitreoretinopathy (EVRX), and advanced retinopathy of prematurity (ROP).

NDP mutations can also cause X-linked form of Familial Exudative Vitreoretinopathy (FEVR) that harbors some of the ND symptoms. FEVR is also caused by mutations in Frizzled4 (Fz4) gene that encodes one of the ten serpentine seven-transmembrane receptors of Wnt-signaling (Robitaille et al., 2002 Nat. Genet. 32: 326-330).

The Wnt family consists of 19 members, and they show high affinity interaction with 10 Frizzled (Fz) proteins. Different Wnts have different affinities for various Frizzled proteins and may engage different pathways (Wu et al., 2002 J. Biol. Chem. 277: 41762-41769). The Wnt-canonical pathway involves beta-catenin stabilization through interaction with Frizzled and its co-receptor lipoprotein related receptor protein 5 or 6 (LRP5/LRP6). In patients and mice, the loss of function mutations in LRP5 also show vascular eye defects in addition to osteoporosis and give rise to osteoporosis-pseudoglioma syndrome (OPPG)(Gong et al., 2001 Cell 207: 513-523; and Kato et al., 2002 J. Cell Biol. 157: 303-314).

Norrin can induce the Wnt-beta-catenin pathway (Xu et al., 2004 Cell 116: 883-895). Norrin functions much like a Wnt, because, both require an Fz receptor and an LRP5/LRP6 co-receptor for signaling, and both bind predominantly to cysteine rich domain (CRD) of Fz with nanomolar affinity (Hsieh et al., 1999 Proc. Nat'l. Acad. Sci. 96: 3546-3551; Wu et al., 2002 J. Biol. Chem. 277: 41762-41769; and Xu et al., 2004 Cell 116: 883-895). Norrin is a cysteine-rich small protein that gets secreted; it forms disulfide-linked oligomers and remains associated with the cell surface and extra cellular matrix (Perez-Vilar et al., 1997 J. Biol. Chem. 272: 33410-33415). From sequence comparisons and modeling studies, it has been suggested that Norrin has a tertiary structure similarity to TGF-beta (Meitinger et al., 1993 Nat. Genet. 5: 376-380), von Willebrand's factor, and mucin (Meindl et al., 1992 Nat. Genet. 2: 139-143). The Norrin gene is expressed predominantly in brain and retina (Berger et al., 1992 Nat. Genet. 1: 199-203; and Chen et al., 1992 Nat. Genet. 1: 204-208).

Drug candidates for treating diseases related to bone remodeling are constantly being sought. The human adult skeleton is in a dynamic state, being continually broken down and reformed by the coordinated actions of osteoclasts and osteoblasts on trabecular (also called cancellous) bone surfaces and in haversian systems. Disruption of bone remodeling can lead to diseases and conditions such as osteoporosis (postmenopausal osteoporosis, glucocorticoid-induced osteoporosis, transplantation induced osteoporosis, and juvenile), rickets, osteomalacia, tumor induced osteomalacia, hypophosphatasia, Paget's disease, and others. Thus, more fully elucidating the pathways controlling bone remodeling and identifying targets in those cascades are useful for developing agents that modulate the targets are needed.

### SUMMARY

The materials and methods described herein provide a greater elucidation of Norrin's involvement in the Wnt pathway via its interaction with LRP5 and 6 and Frizzled4 for bone remodeling and lipid metabolism modulation, and generally provide assays using Norrin and compounds which interact with Norrin (e.g. Norrin agonists) to screen for compounds that are useful in modulating bone disorders and lipid modulation. Also contemplated are methods and materials for identifying Norrin mimetics, as well as other mimetics of the LRP5/Frizzled4/Norrin complex.

Accordingly, an aspect is directed to a method of identifying an agent that modulates bone or a lipid comprising: (a) having a Frizzled4 protein or biologically active Frizzled4 polypeptide with a LRP5 protein or biologically active LRP5 protein in the presence of the agent; and (b) determining whether said agent is a agent that interacts with Frizzled4 and/or LRP5 or a biologically active polypeptide of Frizzled4 or LRP5, and modulates at least one parameter of bone and/or a lipid in the presence of the agent. One aspect has the Frizzled4 protein or biologically active Frizzled4 polypeptide fragment is linked to the LRP5 protein or biologically active polypeptide fragment of LRP5; this can be in the form of a fusion protein. The agent being screened by this method can be a Norrin mimetic, as well as agonists and antagonists of Norrin.

Another aspect is directed to a method of identifying an agent that modulates bone metabolism or lipid metabolism comprising: (a) having a Norrin protein or a biologically active Norrin polypeptide fragment and a Frizzled4 protein or biologically active polypeptide fragment fused to LRP5 and/or LRP6 proteins or biologically active polypeptide fragments thereof in the presence of the agent; and (b) measuring in vitro or in vivo at least one parameter of bone modulation and/or lipid modulation to identify the agent that modulates bone metabolism or lipid metabolism.

The parameters of bone modulation for any of the discussed methods can be any one or more of bone density, bone strength, trabecular number, bone size, or tissue connectivity, or any combination thereof. The parameters of lipid modulation discussed in any of these screening methods can include a change in the level of HDL, VLDL, cholesterol, triglyceride, apoE, or LDL. Another aspect of screening agents in these methods is to see whether they alter expression patterns of genes associated with lipid metabolism or bone metabolism. For example, do they alter expression of one or more of COX-2, Jun, Fos, cyclin D1, Wnt10B, SFRP1, connexin 43, eNOS, Wnt10B, cyclin D1, Frizzled2, and WISP2 is modulated.

The methods can further include Dkk protein or a biologically active Dkk polypeptide fragment and/or a Kremen protein a biologically active Kremen polypeptide fragment and/or a Wnt protein or a biologically active Wnt polypeptide fragment.

In yet another aspect, a method is contemplated for identifying an agent that modulates a Norrin-Frizzled4 activity comprising: (a) having the agent, a Norrin protein or biologically active polypeptide fragment of Norrin, and a Frizzled4 protein or biologically active polypeptide fragment of Frizzled 4 fused to LRP5 and/or LRP6 or a biologically active polypeptide fragment of LRP5 and/or LRP6, or fused to a ligand binding domain (LBD) containing polypeptide fragment of LRP5 or LRP6 and:
(i) a Kremen protein or biologically active polypeptide fragment of Kremen; and/or
(ii) a Dkk protein or biologically active polypeptide fragment of Dkk; and
   (b) determining whether the agent modulates a Norrin-Frizzled4 activity. The agents can be a Norrin mimetic, Norrin agonist, Dkk antagonist, or a Kremen antagonist. It can also be for assessing Frizzled4 agonists and LRP5 agonists.

For certain of these methods, the proteins or biologically active polypeptide fragments of the proteins can be affixed on a substrate, such as PVDF or nitrocellulose.

A further aspect contemplates a method of identifying an agent that regulates bone modulation or lipid modulation comprising: (a) administering the agent to a cell expressing Frizzled4 and LRP5, wherein Frizzled4 is a Frizzled4 protein or biologically active polypeptide fragment of Frizzled4, and LRP5 is a LRP5 protein or a biologically active polypeptide fragment of LRP5; (b) determining whether said administration of the test agent modulates a LRP5-Frizzled4 interaction; and (c) determining whether the agent modulates a bone parameter and/or a lipid parameter. One aspect of this method contemplates that the cell does not express Norrin, which is useful for identifying Norrin mimetics. Another aspect has the cell expressing a non-endogenous Frizzled4, LRP5, LRP6, and/or Norrin and using the cell to, for example, identify Norrin agonists.

Another aspect contemplates a method of identifying an agent that regulates bone modulation or lipid modulation comprising: (a) administering a test agent to a cell expressing LRP5, Norrin and Frizzled4, wherein LRP5 is a LRP5 protein or a biologically active polypeptide fragment of LRP5, Norrin is a Norrin protein or a biologically active Norrin polypeptide, and Frizzled4 is a Frizzled4 protein or a biologically active polypeptide fragment of Frizzled4; (b) determining whether said administration of the test agent modulates Norrin-Frizzled4 interaction; and (c) determining whether the test agent modulates a parameter of bone modulation or lipid modulation. This contemplates the cell optionally expressing a non-endogenous Norrin, LRP5, and/or Frizzled4. Alternatively, the cell may not express an endogenous Norrin, LRP5, LRP6, and/or Frizzled4.

Any of the agents tested can be Norrin mimetics, Dkk antagonists, or Kremen antagonists, as well as Frizzled4 agonists and mimetics, Norrin agonists, and LRP5 agonists.

In any of the methods or cells contemplated the cells can be vertebrate cells. Vertebrate cells can include but are not limited to bone cells, kidney cells, mesenchymal cells, adipocytes, preadipocytes, or Xenopus cells.

In any of the methods, kits, cells/cell lines discussed, the Dkk can be Dkk1, Dkk2, Dkk3, or Dkk4, or a biologically active polypeptide of Dkk1, Dkk2, Dkk3, or Dkk4. Likewise, for Kremen, when Kremen is cited in any aspect it can be Kremen1 or Kremen2, or a biologically active polypeptide of Kremen1 or Kremen2. Also the Wnt can be any of Wnt1 to Wnt19 (e.g., Wnt1, Wnt3, Wnt3a, or Wnt10b) or a biologically active fragment of any of these.

Cell lines for use with any of the methods and kits can include but are not limited to KHOS/NP cells, KHOS-240S cells, KHOS-321H cells, DSDh cells, VA-ES-BJ cells, 7F2 cells, U20S cells, HOSTE85 cells, ROS cells, MC3T3-E6 cells, UMR-106 cells, Saos2 cells, MG63 cells, HOB cells, mesenchymal stem cells (e.g., human adult mesenchymal stem cells), C3H10T1/2 cells, HEK293A cells, or HEK293T cells.

Animals are also contemplated for use in screening the reagents for modulating bone metabolism and lipid metabolism. Animal models include transgenic animals. For example, the animal can be an LRP5 or HBM expressing transgenic animal. Alternatively the animal may be knockout animals wherein one or more of LRP5, LRP5, Norrin, a Dkk, a Kremen, a Wnt, and Frizzled4 are knocked out. Alternatively, the animals also contemplate combined knockouts and introduced genes. The animals can be any vertebrates such as Xenopus or mice.

Yet another embodiment contemplates a kit for identifying an agent which modulates Norrin-Frizzled4 activity comprising: (a) a series of cells incapable of expressing Norrin that are co-transfected with nucleic acids encoding Frizzled4 or a biologically active polypeptide fragment of Frizzled4 and LRP5 or a biologically active polypeptide fragment of LRP5; (b) optionally a Dkk nucleic acid for co-expression in a series of cells co-expressing Frizzled4 or the biologically active polypeptide fragment of Frizzled4 and LRP5 or the biologically active polypeptide fragment of LRP5; (c) optionally a Kremen nucleic acid for co-expression in a series of cells co-expressing Frizzled4 or a biologically active polypeptide fragment of Frizzled4 and LRP5 or a biologically active polypeptide fragment of LRP5, and/or for co-expression in a series of cells co-expressing Frizzled4 or a biologically active polypeptide fragment of Frizzled4, LRP5 or a biologically active polypeptide fragment of LRP5, and Dkk or a biologically active fragment of Dkk; (d) optionally a LRP6 nucleic acid for co-expression in a series of cells co-expressing Frizzled4 or a biologically active polypeptide fragment of Frizzled4 and LRP5 or a biologically active polypeptide fragment of LRP5; and (e) optionally a Wnt nucleic acid for co-expression in a series of cells co-expressing Frizzled4 or a biologically active polypeptide fragment of Frizzled4 and LRP5 or a biologically active polypeptide fragment of LRP5.

Yet another aspect contemplated is a cell or a cell line lacking a native Norrin and which expresses a non-native LRP5 and a non-native Frizzled4, wherein the non-native LRP5 is a non-native LRP5 protein and the non-native Frizzled4 is a non-native Frizzled4 protein, wherein the LRP5 is the complete protein or a biologically active polypeptide fragment of LRP5 and Frizzled4 is the complete protein or a biologically active polypeptide fragment of Frizzled4, and Norrin is the complete protein or a biologically active polypeptide fragment of Norrin. Expression of these proteins can be transient or stable expression. Another aspect contemplates non-native (non-endogenous) expression of LRP5, LRP6, Frizzled4, a Dkk, and/or a Kremen, wherein any of these can be whole protein or biologically active polypeptide fragments thereof.

Another aspect contemplates the agents identified by any one of the methods above alone or in a pharmaceutical composition with suitable pharmaceutically acceptable excipients and/or carriers. The agent can be used to treat a lipid disorder and or a bone disorder or used to formulate a medicament for use in treating one of these disorders.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the materials and methods disclosed and are incorporated in and constitute a part of this specification, illustrate embodiments.

FIG. 1. Norrin activates TCF-signaling in U20S osteoblast-like cells but not in HEK-293A cells. Transient co-transfection assays were conducted in human embryonic kidney (HEK) HEK-293A cells and U20S cells with TCF-luci and renilla reporters. The bar graph represents the ratio of luminescence signals of TCF-luci to renilla signals that normalize the responses between various transfections using different cDNA constructs in pcDNA vector.

FIG. 2. Frizzled4 (Fz4) co-transfection induces Norrin mediated TCF-signal in HEK-293A cells and enhances that in U20S cells. The bar graph shows the results of TCF-luci responses obtained using transient transfections of HEK-293A and U20S cells with various combinations of cDNAs.

FIG. 3. Norrin induced LRP5-Fz4-TCF-signal can be inhibited synergistically by Dkk1 and Kremen2 in U20S cells.

FIG. 4. Norrin mediated TCF signal with LRP5-G171V mutant (HBM phenotype) is less inhibited than that with LRP5 in presence of Dkk1 and Kremen2.

### DETAILED DESCRIPTION

Since it was intriguing that three genes, NDP, Frizzled4 (Fz4), and lipoprotein related receptor protein 5 (LRP5), were found to be involved in vasularization of the retina, investigations on these line revealed that they do interact at the molecular level;

and Norrin is a ligand of LRP5-Fz4 complex. It is interesting to note that the Norrin-mediated activation of LRP5/6 involves Fz4 and not the other five members of the Fz family (i.e., mFZ3, hFZ5, mFz6, mFz7, and mFz8). However, Norrin has specificity to Fz4 and does not show any significant sequence homology to Wnts.

LRP5 mutations in humans and in mice have revealed the pivotal role that LRP5 and Wnt-signal play in bone metabolism (Gong et al., 2001 Cell 207: 513-523; Kato et al., 2002 J. Cell Biol. 157: 303-314; Boyden et al., 2002 N. Engl. J. Med. 346: 1513-1521; and Little et al., 2002 Am. J. Hum. Genet. 70: 1-19). The G171V (high bone mass or “HBM” type) mutation and other such mutations in the first propeller domain of LRP5 results in a decreased affinity of the HBM variants to the Dikkopf1 (Dkk1) protein as compared to that with the wild-type LRP5 (Boyden et al., 2002 N. Engl. J. Med. 346; and Ai et al., 2005 Mol. Cell. Biol. 25: 4946-4955). The HBM mutation leads to decreased inhibition by Dkk1, and the activation of HBM mutant mediated Wnt-beta-catenin signals in vitro. This phenomenon is speculated to be the underlying molecular mechanism important to high bone mass (HBM) phenotype in humans and in transgenic mice with HBM-type mutations (Babij et al., 2003 J Bone Mineral Res. 18: 960-974).

Dkk1 is one of the secreted antagonists of LRP5/6-Wnt signal (Glinka et al., 1998 Nature 391: 357-362; Kawano et al., 2003 J. Cell. Sci. 116: 2627-2634; and Bafico et al., 2001 Nat. Cell Biol. 3: 683-686). In addition, Dkk1 in the presence of Kremen1/2, another type of single pass transmembrane receptor, enhances the inhibition of LRP5/6-TCF signal mediated through Wnt (Mao et. al., 2002 Nature 417: 664-667) by internalization of the LRP5-Dkk1-Kremen ternary complex. Kremens form the ternary complex at the cell surface with LRP5/6 and Dkk1 to facilitate their internalization or endocytosis. Kremens facilitate rapid endocytosis of LRP5 and LRP6 from the cell membrane and thereby block LRP5/6-Wnt signaling. The materials and methods disclosed herein arose from speculation that the expression pattern of these interactors in a given cell type can regulate Wnt-signaling or bring additional specificity to LRP5/6 function in cells, such as osteoblasts. It is to be noted that unless specifically set forth, in all instances wherein Dkk1 is referenced, any of the other Dkks may be substituted alone or in combination.

Analysis of the four splice variants of Kremen2 (Krm2) revealed a variant lacking 44 amino acids at the carboxy terminus, which can enhance Dkk1 mediated inhibition of LRP5/6 (B. Mao et al., “Kremen proteins are Dickkopf Receptors that Regulate Wnt/beta-Catenin Signaling,” 2002 Nature 417(6889): 664-667). Maximal effects of Dkk1 enhancement is seen with a full-length Krm2 clone. Krm2 activity is mediated by its interaction with the second cysteine-rich domain of Dkk1. Krm2 can also convert the LRP6-Wnt signal activator Dkk2, into an inhibitor in HEK-293A cells. It is to be noted that unless specifically set forth, in all instances wherein Kremen2 is referenced, Kremen1 may be substituted alone or in combination with Kremen2.

The materials and methods disclosed herein are directed to the functional interactions between Norrin, Frizzled4, LRP5, or HBM variants of LRP5, e.g., G171V. As described herein, Norrin enhances modestly the TCF-signal of the G171V-LRP5 mutant over the signal observed with LRP5 in U20S bone cells. Norrin also leads to a decreased inhibition of the pathway by Dkk1 and/or Kremen2. Disclosed herein are materials and methods for the use of Norrin as a screening agent for finding reagents that are Norrin mimetics and Norrin agonists. Such Norrin modulating agents and Norrin mimetics may be useful for bone modulation. Norrin modulators and Norrin mimetics include but are not limited to small chemical molecules, polypeptides, peptides, siRNAs, and immunoglobulins.

1. Abbreviations and Definitions

1.1 Abbreviations

The following abbreviations have been used in the specification. Although these acronyms and abbreviations may have different meanings in other arts, they are as indicated below, or as separately distinguished in the specification.
ACP5 acid phosphatase 5
Akt-3 protein kinase B (PKB) or RAC-PK
A1PASE alkaline phosphatase
ALPHA amplified luminescent proximity homogenous assay
APE adaptor-related protein 1
AP1B1 adaptor protein complex AP-1, beta 1 subunit
AXIN axin
b.i.d. bis in die (twice daily)
BGN bone specific biglycan
BMC bone mineral content
BMD bone mineral density

BMP1 bone morphogenetic protein 1
BMP4 bone morphogenetic protein 4
BMU bone remodeling unit
BSA bovine serum albumin
BTG2 B-cell translocation gene 2, anti-proliferative
CBFB core binding factor beta
CCND1 cyclin D1
CCND3 cyclin D3
CCNI cyclin I
cDNA complimentary DNA
CELSR2 cadherin EGF LAG seven-pass G-type receptor 2
CFP cyan fluorescent protein
CHUK/IKK alpha conserved helix-loop-helix ubiquitous kinase, IkB kinase alpha
CK1 alpha casein kinase 1, alpha 1
CKB creatine kinase, brain
CNK1 connector enhancer of KSR-like
Col1A1 collagen, type 1, alpha 1
Col3A1 collagen, type 3, alpha 1
Col6A3 collagen, type VI, alpha 3
Connx43 Connexin 43
COX-2 cyclooxygenase-2
CRABP2 cellular retinoic acid binding protein II
CRD cysteine rich domain
CSF1R colony stimulating factor 1 receptor
CSPG2 chondroitin sulphate proteoglycan
CTGF connective tissue growth factor
CTSK cathepsin K
CX3CR1 chemokine (C—X3-C) receptor 1
Cyclin D1 see also CCND1
DELTEX deltex homolog 2 (Drosophila), see EphB2
Dkk Dikkopf
Dkk1 Dikkopf1
Dkk2 Dikkopf2
Dkk3 Dikkopf3
Dkk4 Dikkopf4
DMSO dimethyl sulphoxide
dsRNA double stranded RNA
DVL1 disheveled, dsh homolog (Drosophila)
DXA dual X-ray absorptiometry
EDTA ethylenediaminetetra acetic acid
EGTA ethylene glycol-O—O′-bis(2-amino-ethyl)-N,N,N′,N′-tetraacetic acid
eNOS excitable nitric oxide synthase
EPHB2 connector enhancer of KSR-like (Drosophila kinase suppressor of ras)
EPHB6 Eph receptor B6
ERBB3 GRO1 oncogene
ERK also known as mitogen activated protein kinase p44/42 (MAPK)
EVRX linked exudative vitreoretinopathy
FAP fibroblast activation protein, alpha
FBLN1 fibulin 1
FBS fetal bovine serum
FEVR familial exudative vitreoretinopathy
FGF-2 Fibroblast growth factor 2 (basic)
FGF-7 Fibroblast growth factor 7 (keratinocyte growth factor)
FOS FBJ murine osteosarcoma viral oncogene homolog
FOSL1 Fos-like antigen 1
FRET fluorescent resonance energy transfer
Frizzled2 Frizzled (Drosophila) homolog 2, also called FZD2
Fz Frizzled
Fz4 Frizzled4
FZD2 Frizzled (Drosophila) homolog 2
FZD4 Frizzled homolog 4
G171V glycine to valine mutation at position 171 of human LRP5
GADD45A growth arrest and DNA-damage inducible, alpha
GADD45B growth arrest and DNA-damage inducible 45, beta
GADD45G growth arrest and DNA-damage inducible 45, gamma
GAS6 growth arrest-specific 6
GFP green fluorescent protein
GJA1 gap junction membrane channel protein alpha 1 (also known as Connexin 43)
GJB3 gap junction membrane channel protein beta 3
GSK-3 glycogen synthase kinase-3
GSK-3α glycogen synthase kinase-3, alpha isoform
GSK-3β glycogen synthase kinase-3, beta isoform
HBM high bone mass phenotype
HDL high density lipoprotein
HEK human embryonic kidney
HERPUDI homocysteine-inducible, endoplasmic reticulum stress-inducible, ubiquitin-like domain member 1
HRT hormone replacement therapy
i.m. intramuscular
i.v. intravenous
IDB2 inhibitor of DNA binding 2
IDB3 insulin-like growth factor 2 (somatomedin A)
IGF2R insulin-like growth factor 2 receptor
IGFBP6 insulin-like growth factor binding protein 6
iGSK GSK inhibitor
iGSK-3 GSK-3 inhibitor
IL-1 interleukin-1
IL1R1 interleukin-1 receptor, type I
IL1RL1 interleukin-1 receptor-like 1
IL4RA interleukin 4 receptor, alpha
IL-6 interleukin-6
ITGA5 integrin alpha 5 (fibronectin receptor alpha)
ITGB5 integrin, beta
ITGBL1 integrin, beta-like 1
JNK c-jun amino kinase pathway
JUN v-jun avian sarcoma virus 17 oncogene homolog
JUND1 Jun proto-oncogene related gene d1
Kremen Kringle coding gene making the eye and the nose
Krm1 Kremen1
Krm2 Kremen2
LBD ligand binding domain of LRP5, LRP6, HBM
LDL low density lipoprotein
LDLR low density lipoprotein receptor
LOX lysyl oxidase
LRP5 low density lipoprotein receptor related protein 5
LRP6 low density lipoprotein receptor-related protein 6
LSP1 lymphocyte-specific protein 1
LUM lumican
mAb monoclonal antibody
MAPK mitogen activated protein kinase (p42,44) (ERK)
MAPKAPK2 mitogen-activated protein kinase-activated protein kinase 2, also called MK2
MCC mutated in colorectal cancers
MDSC mesenchyme derived stem cells
MET met proto-oncogene (hepatocyte growth factor receptor)
MMP-14 matrix metalloproteinase 14
MMP-9 matrix metalloproteinase 9
MSX1 homeo box, msh-like 1
MYBL1 v-myb myeloblastosis viral oncogene homolog (avian)-like 1
MYC v-myc avian myelocytomatosis viral oncogene homolog
MYCS Myc-like oncogene, s-myc protein
NCAM1 neural cell adhesion molecule 1
ND Norrie disease
NDP Norrie disease protein (also Ndph)
NFATC1 nuclear factor of activated T-cells, cytoplasmic 1
NFKB1 nuclear factor of kappa light chain gene enhancer in B-cells 1, p105
Non-TG non-transgenic
NOS3 nitric oxide synthase 3, also known as eNOS
NR4A1 nuclear receptor subfamily 4, group A, member 1
OGN osteoglycin
OPG osteoprotegerin
OPPG osteoporosispseudoglioma syndrome
OSMR oncostatin M receptor
PCOLCE procollagen c-proteinase enhancer protein
PDGFA Cluster Incl. M29464:Platelet derived growth factor alpha
PDGFRA platelet-derived growth factor receptor alpha polypeptide
PKA protein kinase A
PKC protein kinase C
PLAT tissue-type plasminogen activator, t-PA
PNA peptide nucleic acid
PRDC-PENDING protein related to DAC and Cerberus
PTGIS prostaglandin synthase
PTGS post transcriptional gene silencing
PTGS1 prostaglandin-endoperoxide synthase 1, also called COX-1
PTGS2 prostaglandin-endoperoxide synthase 2 (prostaglandin G/H synthase or cyclooxygenase 2) or COX-2
PTH parathyroid hormone
RAMP3 receptor (calcitonin) activity modifying protein 3
RANK receptor activator of NF-kB
RANKL receptor activator of NF-kB ligand
RLUs relative luciferase units
RNAi RNA interference
ROP retinopathy of prematurity
RUNX1 runt related transcription factor 1
RUNX2/CBFA1 runt related transcription factor 2
S100A10 calcium binding protein similar to calpactin
SDC1 syndecan 1
SDF1 stromal derived factor 1
SERM selective estrogen receptor modulator
SERPINE1 serine (or cysteine) proteinase inhibitor, clade E (nexin, plasminogen activator inhibitor type 1), member 1
SFRP1 secreted frizzled-related protein 1
SFRP4 secreted frizzled-related protein 4
shRNA short hairpin RNA
siRNA short interfering RNA
SPARC sparc/osteonectin
SPARCL1 SPARC-like 1 (mast9, hevin)
SPP1 secreted phosphoprotein 1
SPR surface plasmon resonance
STAT1 signal trandsducer and activator of transcription 1
STAT3 RIKEN cDNA 1110034C02 gene
TANK TRAF family member-associated Nf-kappa B activator
TCF T cell factor
TG transgenic
TGFB1 transforming growth factor, beta 1
TGFBR2 transforming growth factor, beta receptor II
TGF-β tumor growth factor beta
THBD thrombomodulin
THBS1 thrombospondin 1
TIEG TGFB inducible early gene
TIMP1 tissue inhibitor of metalloproteinase
TIMP2 tissue inhibitor of metalloproteinase 2
TIMP3 tissue inhibitor of metalloproteinase 3
TNF tumor necrosis factor
TNFRSF10B tumor necrosis factor receptor superfamily, member 10b
TNFRSF11B tumor necrosis factor receptor superfamily, member 11b (Osteoprotegerin)
TNFSF11 tumor necrosis factor (ligand) superfamily, member 11 (see RANKL)
TOB1 transducer of ErbB-2.1
TRAF3 TNF receptor-associated factor 3
TUNEL terminal deoxynucleotidyl transferase dUTP nick end labeling
UNK_D83402 prostaglandin 12 (prostacyclin) synthase
VCAM1 vascular cell adhesion molecule 1
VEH vehicle
VLDL very low density lipoprotein
WIF Wnt inhibitory factor
WISP1 WNT1 inducible pathway protein 1
WISP2 WNT1 inducible signaling pathway protein 2
Wnt 3A wingless-type MMTV integration site family member
Wnt wingless-type MMTV integration site family (e.g., Wnt1 to Wnt 19)

Wnt10B wingless-type MMTV integration site family member 10B
Wnt6 wingless-type MMTV integration site family member 6
YFP yellow fluorescent protein

1.2 Definitions

In accordance with this detailed description, the following abbreviations and definitions apply. It must be noted that as used herein, the singular forms “a”, “an”, and “the” include plural referents unless the context clearly dictates otherwise. Thus, for example, reference to “a mimetic” includes a plurality of such mimetics, and reference to “the dosage” includes reference to one or more dosages and equivalents thereof known to those skilled in the art, and so forth.

Unless defined otherwise, all technical and scientific terms used herein have the same meaning as commonly understood by one of ordinary skill in the art. The following terms are provided below. The genes referred to herein are meant to include the accessions numbers referenced as well as other sequences not specified.

By “animal” is meant any vertebrate. “Animal” includes “mammals.” Preferred mammals include livestock animals (e.g., ungulates, such as cattle, buffalo, horses, sheep, pigs and goats), as well as rodents (e.g., mice, hamsters, rats and guinea pigs), canines, felines, primates (e.g., chimpanzees, orangutans, humans), lupines, camelids, and cervidae. Other vertebrates include avians (e.g., chickens, ducks, geese, and fowl), amphibians (e.g., Xenopus) and ichthyes (fish).

By “Norrin” is meant to include all vertebrate forms of Norrin, and all its polypeptide and nucleic acid forms. “Norrin” is also referred to as ND, Norrie disease protein, Norrin precursor, NDP, Ndp, and Norrie disease protein homology (Ndph). Norrin variants are also contemplated.

By “Norrin activity” would be a Norrin activity as it involves the Wnt signaling pathway and its interaction with Frizzled4 and LRP5/6. This would include Norrin's interaction with Frizzled4 (Fz4) and its enhancement of LRP5 activity. The only Frizzled protein Norrin interacts with is Frizzled4. However, the Wnts discussed herein can be used in the assay systems to compare the specificity of any molecule that would modulate Norrin-Frizzled4 and LRP5 and LRP6 interaction in the Wnt signaling system. Thus, by “Norrin modulating agent” is an agent that modulates a Norrin activity, wherein the Norrin activity is part of Wnt signaling. A preferred Norrin activity is regulation of bone remodeling and/or lipid modulation. With regard to lipid modulation, see U.S. application Ser. No. 09/578,900. The contents of U.S. application Ser. No. 09/578,900 are incorporated herein by reference for all purposes in its entirety. Norrin modulating agents include agonists and antagonists of bone activity and/or lipid levels. A Norrin agonist, for example, would enhance bone growth in a subject when administered.

By “Dkk” is meant to include all vertebrate forms of Dkk1, Dkk2, Dkk3, and Dkk4 and all nucleic acid and polypeptide forms. By “Dkk1” is also referred to as Dickkopf-1, Dickkopf related protein-1 precursor, Dkk-1, DKK-1, hDkk-1 (for the human form of Dkk1), AK, and UNQ492/PRO1008. By “Dkk2” is also meant to include Dickkopf-2, Dickkopf related protein-2 precursor, Dkk-2, DKK-2, hDkk-2 (for the human form of Dkk2), and UNQ682/PRO1316. By “Dkk3” is also meant to include Dickkopf-3, Dickkopf related protein-3 precursor, Dkk-3, hDkk-3 (human form of Dkk3), REIC, and UNQ258/PRO295. By “Dkk4” is meant to include Dickkopf-4, Dickkopf related protein-4 precursor, Dkk-4, DKK-4, and hDkk-4 (for the human form of Dkk4). Dkk variants are also contemplated. Dkk modulating agents would include Dkk antagonists and agonists.

By “Kremen” is meant to include all vertebrate forms of Kremen1 and Kremen2, and all the nucleic acid and polypeptide forms. “Kremen1” is also referred to as Dickkopf receptor, FLJ31863, KREMEN, Kringle-containing protein marking the eye and the nose, Kringle containing transmembrane protein 1, and KRM1. “Kremen2” is also referred to as Dickkopf receptor 2, Kremen protein 2 precursor, Kringle-containing protein marking the eye and the nose, KRM2, MGC10791, MGC16709, and the Kremen2 form associated with the Mammalian Gene Collection (MGC) Program Team, 2002 “Generation and initial analysis of more than 15, 000 full length human and mouse cDNA sequences,” Proc. Nat'l Acad. Sci. USA 99(26): 16899-16903. Kremen variants are also contemplated. Kremen modulating agents would include Kremen antagonists and agonists.

By “LRP5” or “low density lipoprotein receptor-related protein 5” is meant to include all vertebrate nucleic acid and polypeptide forms of LRP5. Other names for LRP5 and related homologs include BMND1, Zmax1, HGNC:8152, low-density lipoprotein receptor-related protein 5 precursor, LR3, LRP7, OPPG, OPS, and VBCH2. “LRP5” is also known as “arr” in Drosophila and has the following additional synonyms: BEST:CK00539, CK00539, 1(2)k08131, LDLR-like, LRP, LRP5/6, and LRP6. For example, one “HBM” or “high bone mass” variant of LRP5 has a single amino acid change in the polypeptide form from a glycine to a valine at position 171 in the human polypeptide sequence. There is a similar mutation at position 170 of the mouse sequence. Additional homologs in other vertebrates can be determined in other species given the three-dimensional propeller domains. Use of HBM contemplates inclusion of the G171V variant and its homolog from other vertebrate species. An HBM variant is one that produces a high bone mass phenotype, which results from a mutation in LRP5 other than the G171V change. For the Zmax1 and HBM forms, see U.S. Pat. No. 6,770,461, which is incorporated herein in its entirety for all purposes. Thus, an example of a wild-type variant or homolog of LRP5 is Zmax1. When reference is made to LRP5, all forms of LRP5 including a wild type variant or homolog are also contemplated. Variants of LRP5 and HBM are also contemplated. LRP5 modulating agents would include agonists and antagonists of LRP5. Also contemplated are LRP5 mimetics.

By “LRP6” or “low density lipoprotein receptor-related protein 6” is meant to include all vertebrate nucleic acid and polypeptide forms of LRP6. LRP6 is also referred to as low-density lipoprotein receptor-related protein 6 precursor. Variants of LRP6 are also contemplated. When reference is made to LRP6, all forms of LRP6 including a wild type variant or homolog are also contemplated. When discussing the Frizzled4/LRP5 complex, the complex is also meant to include Frizzled4/LRP6 and Frizzled4/LRP5/LRP6. LRP6 modulating agents include LRP6 agonists and antagonists. LRP6 mimetics are also contemplated herein for use in modulating the Wnt pathway in a manner to enhance bone growth.

By “Wnt” is meant to include any Wnt (wingless-type MMTV integration site family member) protein and nucleic acid including those of Wnt1-Wnt19. Exemplary Wnt forms include Wnt1 (also known as wingless-type MMTV integration site family member 1, INT1, and Wnt-1 proto-oncogene protein precursor), Wnt3 (also known as wingless-type MMTV integration site family member 3, INT4, and Wnt-3 proto-oncogene protein precursor), Wnt3a (also known as wingless-type MMTV integration site family member 3A and Wnt-3a protein precursor), and Wnt10b (also known as wingless-type MMTV integration site family member 10B, Wnt-10b protein precursor, WNT-12, Wnt-12, and WNT-12). Variants of any of the Wnt forms are also contemplated. Wnt modulating agents include Wnt agonists and antagonists.

By “variant” is meant to include a form of a nucleic acid encoding a protein, wherein the protein has biological activity in the Wnt cascade, and is involved in modulation of bone metabolism and lipid metabolism. This can include augmented variants of LRP5, such as the G171V variant that produces a high bone mass in the human expressing this protein.

By “biologically active fragment”, “polypeptide fragment”, and “biologically active polypeptide” are meant a biologically active fragment of LRP5, LRP6, HBM, Kremen1, Kremen2, any Dkk, any Wnt, and Norrin, wherein such activity modulates the Wnt pathway, and preferably the Wnt pathway with regard to bone development, bone modulation, and/or metabolism of a lipid. These are domains of the complete proteins that are involved with Wnt signaling, and thereby Wnt pathway induced modulation of lipids and/or bone development. For example, biologically active polypeptides of LRP5 and LRP6 can be the extracellular portion of those proteins (e.g., amino acids 1-1376 of human LRP5 (GenBank Accession No. NP_{—}002326), Zmax1, or HBM). Additionally, for human LRP5, which is 1615 amino acids in length, other domains with biological activity may can include the transmembrane domain (amino acids 1385 to 1407), the cytoplasmic domain (amino acids 1408 to 1615), and the extracellular domain (amino acids 1-1384 or 20-1384 if the first 19 amino acids of the signal peptide are removed). For human LRP6, which is 1613 amino acids long, would have analogous domains: extracellular domain (amino acids 1-1370 or 20-1370 if the first 19 amino acids of the signal peptide are removed), transmembrane domain (amino acids 1371-1393), and the cytoplasmic domain (amino acids 1394-1613). The extracellular cysteine rich domain (CRD) of Frizzled4 has been shown to interact with Norrin, i.e., amino acids 36-165 (Accession No. IPR000024; GenBank Accession No. NP_{—}036325; Xu et al., 2004 Cell 116: 883-895); thus a biologically active polypeptide of Frizzled4 could contain the CRD. A biologically active polypeptide of Norrin could include the CRD domain of Norrin, e.g., amino acids 15-150. In another example, it has been reported that for a Dkk protein to bind to Kremen1 or Kremen2, the entire extracellular domain is required, e.g., amino acids 1-362 for human Kremen2 (GenBank Accession No. BAC00872). Thus, biologically active portions of Kremen1 and Kremen2 would contain at least the extracellular domain, as well as longer sequences thereof. For Dkk1, for example, a biologically active polypeptide would contain at least the C-terminal cysteine rich domain (amino acids 183-266 for human Dkk1; GenBank Accession No. AAQ89364). It is known that the C-terminal cysteine rich domain is involved in the binding of LRP5 and LRP6 to Kremen2. Thus, for any biologically active polypeptide of a Dkk protein, the polypeptide could contain at least the cysteine rich domain of each Dkk. However, other examples include polypeptides containing the cysteine rich domain of a Dkk protein as well as, for example in Dkk1, sequences both to the N-terminal and/or C-terminal ends of the cysteine rich domain of Dkk1. Similar sequences would be contemplated for the other Dkks. Such biologically active fragments can also include complete proteins minus one or more amino acids at either the carboxy terminus, or amino terminus, or within the polypeptide that forms the protein, but which have the same activity as the full-length protein and wherein such biologically active polypeptide fragments do not act as blocking inhibitor when compared with activity induced by the full-length polypeptide.

By a “lipid parameter” is meant to include, but is not limited to an in vitro or in vivo measured parameter to analyze a change of lipid concentration based on exposure to a reagent. The lipid parameter can include measurement of apoE, HDL, LDL, VLDL, triglyceride, cholesterol, the number of adipocytes, a change in adipocyte gene expression, or a combination of these parameters. A lipid parameter is also meant to include ratios of, for example HDL:VLDL. If studying in vivo changes, lipid profiles can be done such as fasting lipid profiles (total cholesterol, triglycerides, LDL and HDL) to assess modulation of lipid levels due to administration of a test reagent.

By “lipid disorders”, “lipid diseases,” and “lipid conditions” which may be mediated by Norrin are meant to include but are not limited to familial lipoprotein lipase deficiency, familial apoprotein CII deficiency, familial type 3 hyperlipoproteinemia, familial hypercholesterolemia, familial hypertriglyceridemia, multiple lipoprotein-type hyperlipidemia, elevated lipid levels due to dialysis and/or diabetes, and elevated lipid levels of unknown etiologies.

“Bone development” generally refers to any process involved in the change of bone over time, including, for example, normal development, changes that occur during a disease state, and changes that occur during aging or changes in hormonal pattern. This may refer to structural changes and dynamic rate changes such as growth rates, resorption rates, bone repair rates, and etc. “Bone development disorder” particularly refers to any disorders in bone development including, for example, changes that occur during disease states and changes that occur during aging. Bone development may be progressive or cyclical in nature. Aspects of bone that may change during development include, for example, mineralization, formation of specific anatomical features, and relative or absolute numbers of various cell types. Other bone disorders contemplated that may not be tied to development include but are not limited to age related loss of bone, bone fractures (e.g., hip fracture, Colle's fracture, vertebral crush fractures), chondrodystrophies, drug-induced disorders (e.g., osteoporosis due to administration of glucocorticoids or heparin, and osteomalacia due to administration of aluminum hydroxide, anticonvulsants, or glutethimide), high bone turnover, hypercalcemia, hyperostosis, osteogenesis imperfecta, osteomalacia, osteomyelitis, osteoporosis, Paget's disease, osteoarthritis, and rickets.

“Bone modulation” or “modulation of bone formation” refers to the ability to affect any of the physiological processes involved in bone remodeling, as will be appreciated by one skilled in the art, including, for example, bone resorption and appositional bone growth, by amongst other things, osteoclastic and osteoblastic activity, and may comprise some or all of bone formation and development as used herein.

Bone is a dynamic tissue that is continually adapting and renewing itself through the renewal of old or unnecessary bone by osteoclasts and the rebuilding of new bone by osteoblasts. The nature of the coupling between these processes is responsible for both the modeling of bone during growth as well as the maintenance of adult skeletal integrity through remodeling and repair to meet the everyday needs of mechanical usage. There are a number of diseases that result from an uncoupling of the balance between bone resorption and formation. With aging there is a gradual “physiologic” imbalance in bone turnover, which is particularly exacerbated in women due to menopausal loss of estrogen support that leads to a progressive loss of bone. As bone mineral density falls below population norms, there is a consequential increase in bone fragility and susceptibility to spontaneous fractures. For every 10 percent of bone that is lost, the risk of fracture doubles. Individuals with bone mineral density (BMD) in the spine or proximal femur 2.5 or more standard deviations below normal peak bone mass are classified as osteoporotic. However, osteopenic individuals with BMD between 1 and 2.5 standard deviations below the norm are also at risk.

Bone is measured by several different forms of X-ray absorptiometry. All of the instruments measure the inorganic or bone mineral content of the bone. Standard DXA measurements give a value that is an areal density, not a true density measurement by the classical definition of density (mass/unit volume). Nevertheless, this is the type of measurement used clinically to diagnose osteoporosis. However, while BMD is a major contributing factor to bone strength, as much as 40% of bone strength stems from other factors including but not limited to: (1) bone size (i.e., larger diameters increase organ-level stiffness, even in the face of lower density); (2) the connectivity of trabecular structures; (3) the level of remodeling (remodeling loci are local concentrators of strain); and (4) the intrinsic strength of the bony material itself, which in turn is a function of loading history (i.e., through accumulated fatigue damage) and the extent of collagen cross-linking and level of mineralization. There is good evidence that all of these strength/fragility factors play some role in osteoporotic fractures, as do a host of extraskeletal influences as well (such as but not limited to fall patterns, soft tissue padding, and central nervous system reflex responsiveness).

Additional analytical instruments can be used to address these features of bone. For example, the pQCT allows measurement of separate trabecular and cortical compartments for size and density. The μCT (micro CT) provides quantitative information on architectural features such as trabecular connectivity. The μCT also gives a true bone density measurement. With these tools, the important non-BMD parameters can be measured for diagnosing the extent of disease and the efficacy of treatments. Current treatments for osteoporosis are based on the ability of drugs to prevent or retard bone resorption. Although newer anti-resorptive agents are proving to be useful in the therapy of osteoporosis, they are viewed as short-term solutions to the more definitive challenge to develop treatments that will increase bone mass and/or the bone quality parameters mentioned above. Thus, bone modulation may be assessed by measuring parameters such as bone mineral density (BMD) and bone mineral content (BMC) by pDXA X-ray methods, bone size, thickness or volume as measured by X-ray, bone formation rates as measured, for example, by calcien labeling, total, trabecular, and mid-shaft density (as measured by pQCT and/or μCT methods), connectivity and other histological parameters (as measured by μCT methods), mechanical bending and compressive strengths (as preferably measured in femur and vertebrae respectively). Thus, measurable parameters include but are not limited to bone density, bone strength, trabecular number, bone size, and bone tissue connectivity. Due to the nature of these measurements, each may be more or less appropriate for a given situation as the skilled practitioner will appreciate. Furthermore, parameters and methodologies such as a clinical history of freedom from fracture, bone shape, bone morphology, connectivity, normal histology, fracture repair rates, and other bone quality parameters are known and used in the art. Most preferably, bone quality may be assessed by the compressive strength of vertebra when such a measurement is appropriate. Bone modulation may also be assessed by rates of change in the various parameters. Most preferably, bone modulation is assessed at more than one age. Compounds can be assessed over any one or more of the parameters listed herein for determining modulation of bone density.

“Normal bone density” refers to a bone density within two standard deviations of a Z score of 0 in the context of the HBM linkage study. In a general context, the range of normal bone density parameters is determined by routine statistical methods. A normal parameter is within about 1 or 2 standard deviations of the age and sex normalized parameter, preferably about 2 standard deviations. A statistical measure of meaningfulness is the P value which can represent the likelihood that the associated measurement is significantly different from the mean. Significant P values are P<0.05, 0.01, 0.005, and 0.001, preferably at least P<0.01.

The terms “force”, “load”, “stress” and “strain” are used interchangeably herein and are related to the principles of force, which in mechanics is any action that tends to maintain or alter the position of a body or to distort it and this term is used interchangeably with load in this document. Force as a measure per unit area is defined as “stress,” and is also referred to herein as “mechanical stress” and can be classified as compressive, tensile or shear depending on how the forces (load) are applied. Specifically, compressive stresses are developed if loads are applied so that the material becomes shorter, whereas tensile stresses are developed when the material is stretched. Shear stresses are developed when one region of a material slides relative to an adjacent region. The result of stress is defined as deformation and the percentage of the relative deformation or change in length is termed “strain”. If for example a material is stretched to 101% of its original length it has a strain of 0.01 or 1%. Since strain has no units it is either reported as relative deformation where a strain of 0.01 is equal to 1% deformation or in terms of microstrain where 10,000 microstrain is equal to 0.01 strain or 1% deformation (Turner et al., 1993 Bone, 14: 595-608).

By “test agent,” and “test reagent” is meant to include small compounds, compositions, peptides, mimetics, polypeptides, siRNAs, and immunoglobulins. Compositions include combinations of two or more active compounds, wherein one or more of the active compounds are Wnt pathway (cascade) modulators.

By “immunoglobulins” is meant to include antibodies and antibody fragments. As used herein, the term “antibody” is meant to refer to complete, intact antibodies, diabodies, and antibody fragments such as Fab fragments, Fab′, and F(ab)₂ fragments. Complete antibodies include monoclonal antibodies (mAb), such as murine monoclonal antibodies, chimeric antibodies, humanized antibodies, primatized antibodies, and human antibodies. The production of antibodies and genetically engineered or enzymatically produces portions of antibodies and the organization of the genetic sequences that encode such molecules are well known and are described, for example, in Harlow et al., ANTIBODIES: A LABORATORY MANUAL, Cold Spring Harbor Laboratory, Cold Spring Harbor, N.Y. (1988); Harlow et al., USING ANTIBODIES: A LABORATORY MANUAL, (Cold Spring Harbor Press, New York, 1998); and Breitling et al., RECOMBINANT ANTIBODIES (Wiley-Spektrum, 1999), which are incorporated herein by reference for all purposes. Immunoglobulins also include fragments such as scFv.

By “immunologically active” is meant any immunoglobulin protein or fragment thereof which recognizes and binds to an antigen. Preferably, the immunologically active protein or fragment thereof modulates the antigen to which it binds. For example, if it binds to Norrin or to a ligand of Norrin, the immunologically active protein or fragment thereof would modulate Norrin activity or the activity of the Norrin ligand.

“Single-chain Fvs” (“scFvs”) are recombinant antibody fragments consisting of only the variable light chain (V_{L}) and variable heavy chain (V_{H}) covalently connected to one another by a polypeptide linker. Either V_{L} or V_{H} may be the NH₂-terminal domain. The polypeptide linker may be of variable length and composition so long as the two variable domains are bridged without serious steric interference. Typically, the linkers are comprised primarily of stretches of glycine and serine residues with some glutamic acid or lysine residues interspersed for solubility.

“Diabodies” are dimeric scFvs. The components of diabodies typically have shorter peptide linkers than most scFvs, and they show a preference for associating as dimers.

An “Fv” fragment is an antibody fragment that consists of one V_{H} and one V_{L} domain held together by non-covalent interactions. The term “dsFv” is used herein to refer to an Fv with an engineered intermolecular disulfide bond to stabilize the V_{H}-V_{L} pair.

A “F(ab′)₂” fragment is an antibody fragment essentially equivalent to that obtained from immunoglobulins (typically IgG) by digestion with the enzyme pepsin at pH 4.0-4.5. The fragment may also be recombinantly produced.

A “Fab” fragment is an antibody fragment essentially equivalent to that obtained by reduction of the disulfide bridge or bridges joining the two heavy chain pieces in the F(ab′)₂ fragment. The Fab′ fragment may also be recombinantly produced.

The term “protein-capture agent” means a molecule or a multi-molecular complex, which can bind a protein to itself. Protein-capture agents preferably bind their binding partners in a substantially specific manner. Protein-capture agents with a dissociation constant (K_{D}) of less than about 10⁻⁶ are preferred (e.g., 10⁻⁷, 10⁻⁸, 10⁻¹⁰). Antibodies or antibody fragments are highly suitable as protein-capture agents. Antigens may also serve as protein-capture agents, since they are capable of binding antibodies. A receptor that binds a protein ligand is another example of a possible protein-capture agent. Protein-capture agents are understood not to be limited to agents, which only interact with their binding partners through non-covalent interactions. Protein-capture agents may also optionally become covalently attached to the proteins, which they bind. For instance, the protein-capture agent may be photo-crosslinked to its binding partner following binding.

The term “binding partner” means a protein that is bound by a particular protein-capture agent, preferably in a substantially specific manner. In some cases, the binding partner may be the protein normally bound in vivo by a protein that is a protein-capture agent. In other embodiments, however, the binding partner may be the protein or peptide on which the protein-capture agent was selected (through in vitro or in vivo selection) or raised (as in the case of antibodies). A binding partner may be shared by more than one protein-capture agent. For instance, a binding partner that is bound by a variety of polyclonal antibodies may bear a number of different epitopes. One protein-capture agent may also bind to a multitude of binding partners (for instance, if the binding partners share the same epitope).

“Conditions suitable for protein binding” means those conditions (in terms of salt concentration, pH, detergent, protein concentration, temperature, etc.) which allow for binding to occur between a protein and its binding partner in solution. Preferably, the conditions are not so lenient that a significant amount of non-specific protein binding occurs.

An “array” is an arrangement of entities in a pattern on a substrate. Although the pattern is often a two-dimensional pattern, the pattern may also be a three-dimensional pattern for a greater application of the material to the array substrate.

The term “substrate” refers to the bulk, underlying, and core material of the arrays of the invention. The substrate is the material to which nucleic acids, antibodies, immunoglobulins and other compounds are affixed.

By “transgenic animal” is meant an animal harboring in its germ line a gene or nucleic acid that has been introduced by cDNA technology. This can be, for examples, introduction of human genes into rodents or a mouse gene in a mouse. The term can include knock-out animals and knock-in animals and combinations for example wherein an animal has had its wild-type gene knocked out and then replaced. The replaced gene can be the native wild-type gene, a cognate gene from another animal such as a human gene, or a variant such as LRP5. The introduced gene can also be under control of an inducible promoter. The HBM variant cDNA can be a native variant or non-native variant. For example, the human HBM variant of G171V can be introduced into a mouse. Alternatively, the mouse counterpart to G171V can also be introduced into a mouse yielding a transgenic HBM mouse expressing a native HBM variant. A transgenic animal is not meant to include transgenic humans, but can include non-human primates and other animals. The transgenic animal can have knocked out or introduced any one or more Dkk, Norrin, LRP5, LRP6, Kremen, Wnt, or Frizzled4. A transgenic animal is contemplated to be a non-human animal, but can include non-human primates.

By “LRP5 transgenic animal” is means to include an animal expressing both the native and a cDNA form of LRP5 or only a cDNA form of LRP5 if the animal has the native form of LRP5 removed or incapable of function (knocked out). The cDNA form of LRP5 may be under an inducible element. The animal can be one wherein the native gene is knocked out and a native or non-native LRP5 has been introduced, or knocked-in. These knock-in animals again can have the genes preferably under inducible control.

By an “HBM transgenic animal” is meant an animal wherein the native LRP5 is present or knocked out and a cDNA encoding the HBM variant is present.

By “effective amount” or “dose effective amount” or “therapeutically effective amount” is meant an amount of an agent which modulates a biological activity of Norrin sufficient to modulate a bone parameter and/or a lipid parameter.

The term “recognizes and binds,” when used to define interactions of antisense nucleotides, siRNAs (small inhibitory RNA), or shRNAs (short hairpin RNAs) with a target sequence, means that a particular antisense, siRNA, or shRNA sequence is substantially complementary to the target sequence, and thus will specifically bind to a portion of an mRNA encoding polypeptide. As such, typically the sequences will be highly complementary to the mRNA target sequence, and will have no more than 1, 2, 3, 4, 5, 6, 7, 8, 9, or 10 base mismatches throughout the sequence. In many instances, it may be desirable for the sequences to be exact matches, i.e. be completely complementary to the sequence to which the oligonucleotide specifically binds, and therefore have zero mismatches along the complementary stretch. Highly complementary sequences will typically bind quite specifically to the target sequence region of the mRNA and will therefore be highly efficient in reducing, and/or even inhibiting the translation of the target mRNA sequence into polypeptide product.

Substantially complementary oligonucleotide sequences will be greater than about 80 percent complementary (or “% identity”) to the corresponding mRNA target sequence to which the oligonucleotide specifically binds, and will, more preferably be greater than about 85 percent complementary to the corresponding mRNA target sequence to which the oligonucleotide specifically binds. In certain aspects, as described above, it will be desirable to have even more substantially complementary oligonucleotide sequences for use in the practice of the invention, and in such instances, the oligonucleotide sequences will be greater than about 90 percent complementary to the corresponding mRNA target sequence to which the oligonucleotide specifically binds, and may in certain embodiments be greater than about 95 percent complementary to the corresponding mRNA target sequence to which the oligonucleotide specifically binds, and even up to and including 96%, 97%, 98%, 99%, and even 100% exact match complementary to the target mRNA to which the designed oligonucleotide specifically binds.

Percent similarity or percent complementary of any of the disclosed sequences may be determined, for example, by comparing sequence information using the GAP computer program, version 6.0, available from the University of Wisconsin Genetics Computer Group (UWGCG). The GAP program utilizes the alignment method of Needleman and Wunsch, 1970 J. Mol. Biol. 48(3): 443-53. Briefly, the GAP program defines similarity as the number of aligned symbols (i.e., nucleotides or amino acids) which are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986 Nucleic Acids Res. 14(1): 327-34), (2) a penalty of 3.0 for each gap and an additional 0.10 penalty for each symbol in each gap; and (3) no penalty for end gaps.

By “mimetic” is meant a molecule that performs the same function or behaves similarly to the mimicked agent or has an activity that is enhanced to that of the agent being mimicked. For example, a Norrin mimetic would interact with LRP5 and/or LRP6 and Frizzled4 as the Norrin polypeptide does and modulate bone mass and/or lipid levels like Norrin or at an enhanced level to that observed for Norrin. For example, the mimetic could induce a high bone mass like phenotype, such as observed for the HBM phenotype (which results for example from the G171V mutation in the human LRP5 polypeptide, or the cognate location in another vertebrate LRP5). The mimetic molecule can be a polypeptide, peptide, immunoglobulin, or a small chemical compound.

By “reporter element” is meant a polynucleotide that encodes a polypeptide capable of being detected in a screening assay. Examples of polypeptides encoded by reporter elements include, but are not limited to, lacZ, GFP, YFP (or other fluorescent reporter), luciferase, and chloramphenicol acetyltransferase.

By “cell” or “host cell” is meant to include vertebrate cells, as well as yeast cells or certain prokaryotic cells for use in screening assays. For example, a suitable cell may be a yeast cell in a yeast two hybrid assay.

By “bone cell” is meant to include cells from tissue culture (“cultured cell”) or cells obtained from bone tissue. Such cells include but are not limited to osteoblasts, preosteoblasts, osteoprogenitor cells, osteoclasts, osteocytes, mesenchymal stem cells, any of the cells discussed herein, or any combination thereof. By bone tissue would mean to include a combination of these cells, as may be obtained from a bone biopsy.

By “Dkk antagonist” is meant to include but not limited to monoclonal or polyclonal antibodies or immunogenically active fragments thereof, peptide aptamers, a GSK binding protein, an antisense molecule to a GSK nucleic acid, an RNA interference molecule, a morpholino oligonucleotide, a peptide nucleic acid (PNA), a ribozyme, and a peptide that can inhibit Dkk activity in the Wnt pathway.

Likewise, by “Kremen antagonist” is meant to include but not limited to monoclonal or polyclonal antibodies or immunogenic active fragments thereof, peptide aptamers, an RNA interference molecule, a morpholino oligonucleotide, a peptide nucleic acid (PNA), a ribozyme, and a peptide that can inhibit Kremen activity in the Wnt pathway.

By “Wnt 3A agonist” is meant to include reagents which can up regulate Wnt 3A synthesis and/or activity. By “Wnt 3A mimetic” is meant a molecule that mimics Wnt3A activity. By “Wnt 3A variant” would include any functional variant which when administered with load can enhance activation with a Wnt/β-catenin response.

The term “fusion protein” refers to a protein composed of two or more polypeptides that, although typically not joined together in their native state, are joined by their respective amino and carboxyl termini through a peptide linkage to form a single continuous polypeptide. It is understood that the two or more polypeptide components can either be directly joined or indirectly joined through a peptide linker/spacer.

2. Assays for Screening Test Agents Which Modulate Norrin

The materials and methods disclosed herein are directed in part to methods of screening agents that modulate NDP genes or the Norrin proteins encoded by those genes or identifying Norrin mimetics and Norrin agonists. The assays are also directed to materials and methods of screening agents that modulate reagents that interact with Norrin proteins, identifying Norrin agonists, and identifying Norrin mimetics. These could be reagents that by binding with Frizzled4, modulate Norrin activity. These can also be reagents which by modulating Dkk1 activity (either the gene or the protein) modulate Norrin activity. Another example would be Kremen2, wherein modulation of Kremen2 (either the gene or the protein) would modulate Norrin activity. In the instances of Dkk1 and Kremen2, preferably the reagent modulating the activity of these compounds would be an antagonist of Dkk1 or Kremen2. Preferably, the assays would result in reagents that also modulate LRP5 activity via Frizzled4 and Norrin interaction. Preferable LRP5 modulation would be in the form of enhanced activity, such as that produced by an agonist, or a mimetic (e.g., a Norrin mimetic or Frizzled4 mimetic).

Assay systems can include a step wherein test agents are screened for their ability to bind to Frizzled4, Norrin, Dkk1, Kremen2, LRP5, or act as a Norrin mimetic. This can be any system both involving substrates or free in solution, wherein binding of the test agents to any of the aforementioned substrates is allowed to occur and then assayed to determined binding. Test agents can be admixed with Frizzled4, Norrin, Dkk1, Kremen2, or LRP5 under physiological conditions (e.g., pH about 7.0 to about 7.4; 24° C. to about 40° C.) for a sufficient period of time to permit binding, e.g., about 1 minute to 6 hours.

Test agents can be screen for binding as discussed above or can be candidates from any chemical library. The test agents can then be assayed in a cell-based assay system. Such a cell-based assay system can be one wherein the cells are transiently or stably transfected with a nucleic acid encoding at least one of the following: Frizzled4, Norrin, Dkk1, Kremen2, or LRP5, or any combination thereof. Thus, cells would individually express at least Frizzled4 and Norrin, as well as the remaining three genes. There would also be a series of cells stably or transiently co-transfected with the following combinations of nucleic acids:

(a) Norrin and LRP5 and/or LRP6

(b) Norrin, a Dkk (e.g., Dkk 1 to Dkk4) and LRP5 and/or LRP6

(c) Norrin, a Kremen (e.g., Kremen 1 or 2) and LRP5 and/or LRP6

(d) Norrin, a Kremen, a Dkk, and LRP5 and/or LRP6

(e) Frizzled4 and Norrin;

(f) Frizzled4, Norrin, and LRP5

(g) Frizzled4, Norrin, and a Dkk (e.g., Dkk1 to Dkk4);

(h) Frizzled4, Norrin, and a Kremen (e.g., Kremen 1 and/or 2);

(i) Frizzled4, Norrin, a Dkk, and Kremen2;

(j) Frizzled4, Norrin, LRP5, and Dkk1;

(k) Frizzled4, Norrin, LRP5, and Kremen2; and/or

(l) Frizzled4, Norrin, LRP5, Dkk1, and Kremen2, and/or

(m) or any combination.

Also contemplated for any of the above combinations are LRP6, HBM, other Dkks (e.g., Dkk2, Dkk3, and/or Dkk4), Wnts, and/or Kremen1.

It would be understood by one of ordinary skill that such an assay system may also require vector controls, wherein the vector is that in which the nucleic acid encoding any of the above proteins is operably linked for expression in the cells. The vector control can consist of the transient or stable transfection of cells with only the vector and/or with no vector. Transient transfection and stable transfection of cells can be performed using techniques known in the art. See, e.g., Sambrook et al., MOLECULAR CLONING: A LABORATORY MANUAL, volumes 1-3 (3^{rd} ed., Cold Spring Harbor Press, NY 2001) or any of the prior editions by Sambrook. Co-transfections can be prepared either transiently or stably as known in the art. Sambrook et al., 2001

Nucleic acids encoding Frizzled4, Norrin, LRP5, Dkk1, and Kremen are listed in part below along with their associated protein sequences. The embodiments of this application are not limited to the sequences disclosed herein.

| | | | Nucleic Acid |
|---|---|---|---|
| | | Protein GenBank | GenBank |
| Gene | Organism | Accession No. | Accession No. |
| Norrin | Homo sapiens | AAH29901 | BC029901.1 |
| | Homo sapiens | CAA46639 | X65724.1 |
| | Mus musculus | AAH90623 | BC090623 |
| | Mus musculus | CAA58725 | X83794.1 |
| Dkk1 | Homo sapiens | AAF02674 | AF177394.1 |
| | Mus musculus | AAH50189 | BC050189.1 |
| | Mus musculus | AAC02426.1 | AF030433.1 |
| | Danio rerio | BAA82135 | AB023488.1 |
| | Danio rerio | AAD2246.1 | AF116852.1 |
| Dkk2 | Homo sapiens | AAF02675 | AF177395 |
| | Homo sapiens | AAH75078 | BC075078.2 |
| | Mus musculus | CAP6010.1 | AJ243963.2 |
| Dkk3 | Homo sapiens | AAF02676 | AF177396.1 |
| | Homo sapiens | AAH07660 | BC007660.2 |
| | Homo sapiens | AAQ88744 | AY358378.1 |
| | Mus musculus | AAF02680 | AF177400.1 |
| | Mus musculus | AAH46304 | BC046304.1 |
| | Mus musculus | AAH50934 | BC050934.1 |
| Dkk4 | Homo sapiens | AAF02677 | AF177397.1 |
| | Homo sapiens | BAA33438.1 | AB01778 |
| | Mus musculus | AAH18400 | BC018400 |
| Frizzled4 | Homo sapiens | BAA86286.1 | AB032417.1 |
| | Homo sapiens | BAB40811.1 | AB054881 |
| | | FZD4S splicing | |
| | | variant | |
| | Homo sapiens | AAR23924.1 | AY462097.1 |
| | Mus musculus | AAH15256 | BC015256.1 |
| | Mus musculus | AAC52430 | U43317.1 |
| | Drosophila | AAF81195 | AF241270.1 |
| | melanogaster | | |
| LRP5 | Homo sapiens | AAC36467.1 | AF064548.1 |
| | Homo sapiens | AAC72791 | AF077820.1 |
| | Homo sapiens | AAK52433 | AF283320.1 |
| | Mus musculus | AAC36468 | AF064984.1 |
| | Mus musculus | AAC70183 | AF077847.1 |
| | Mus musculus | AAH11374 | BC011374.1 |
| | Drosophila | AAF58373 | AE003818.3 |
| | melanogaster | | |
| LRP6 | Homo sapiens | AAC33006 | AF074264.1 |
| | Mus musculus | AAC33007 | AF074265.1 |
| | Mus musculus | AAH60704 | BC060704.1 |
| | Drosophila | AAF58373 | AE003818.3 |
| | melanogaster (arr) | | |
| Kremen1 | Homo sapiens | AAH63787 | BC063787.1 |
| | Homo sapiens | BAB40969.1 | AB059618 |
| | Mus musculus | BAB40968.1 | AB059617 |
| | Mus musculus | | BC049771 |
| Kremen2 | Homo sapiens | AAH03533 | BC003533.1 |
| | Homo sapiens | AAH09383 | BC009383.2 |
| | Homo sapiens | BAC00823.1 | AB086355 |
| | Mus musculus | CAD29805 | AJ457192 |

Additional HBM and LRP5 sequences (e.g., Zmax1) are disclosed in U.S. application Ser. Nos. 09/544,398 (now U.S. Pat. No. 6,770,461) and 10/240,851, which are herein incorporated by reference in their entirety for all purposes.

The cells which can be transfected can be any mammalian cell line. Preferable cell lines are human cell lines, especially when using nucleic acids which encode human proteins for any of the above. Thus, transfections can occur for mouse nucleic acids in mouse lines or for human nucleic acids in human lines. Cell lines can be bone cell lines, kidney cell lines stem cell lines from humans or other vertebrates. Exemplary kidney cell lines include but are not limited to HEK-293 cells (ATCC® No. CRL-1573) and HepG2 cells. Exemplary bone cell lines include but are not limited to KHOS/NP (R-970-5) (ATCC® No. CRL-1544), KHOS-240S (ATCC® No. CRL-1545), KHOS-321H (ATCC® No. CRL-1546), DSDh (ATCC® No. CRL-2131), VA-ES-BJ (ATCC® No. CRL-2138), 7F2 (ATCC® No. CRL-12557), U-2 OS (also known as U20S; ATCC® No. HTB-96), HOSTE85, ROS, MC3T3-E6, UMR-106, Saos2, MG63, and HOBs. Exemplary stern cell lines include but are not limited to human adult mesenchymal stem cells (Cambrex Bioscience) and the mouse stem cell line, C3H10T1/2 (ATCC).

The nucleic acids encoding any of the proteins would include the open reading frames (ORFs), as well as any transcriptional information necessary for transcription and translation. The nucleic acids encoding the proteins would in turn be operably linked to a vector suitable for stable and/or transient transfection in a cell. Suitable vectors include but are not limited to TK-renilla, pcDNA3.1 (Invitrogen), and pUSE (Upstate Biotech). Other operable vectors capable of expression in vertebrate cells may be used.

Any reporter system that provides information on the regulation of genes and their associated proteins can be utilized, including but not limited to TK-renilla, β-galactosidase (β-gal), alkaline phosphatase, green fluorescent protein (GFP), or other fluorescent protein marker. A preferred system, as described herein, is the combination of TCF-luci and TK-renilla as described in the examples. Other reporter and vector combinations operative in vertebrate cells may also be utilized.

In one aspect, the relative amounts of Norrin or a Norrin interacting protein of a cell population that has been exposed to the agent to be tested is compared to an un-exposed control cell population. Antibodies can be used to monitor the differential expression of the protein in the different cell populations. Cell lines or populations are exposed to the agent to be tested under appropriate conditions and time. Cellular lysates may be prepared from the exposed cell line or population and a control, unexposed cell line, or population. The cellular lysates are then analyzed with the probe, as would be known in the art. See, e.g., Ed Harlow and David Lane, ANTIBODIES: A LABORATORY MANUAL (Cold Spring Harbor, N.Y., 1988) and Ed Harlow and David Lane, USING ANTIBODIES: A LABORATORY MANUAL (Cold Spring Harbor, N.Y. 1998).

For example, N- and C-terminal fragments of Norrin can be expressed in bacteria and used to search for proteins which bind to these fragments. Fusion proteins, such as His-tag or GST fusion to the N- or C-terminal regions of Norrin (or to biologically active domains of Norrin) or a whole Norrin protein can be prepared. These fusion proteins can be coupled to, for example, Talon or Glutathione-Sepharose beads and then probed with cell lysates to identify molecules which bind to Norrin. Prior to lysis, the cells may be treated with purified Wnt proteins, RNA, or drugs which may modulate Wnt signaling or proteins that interact with downstream elements of the Wnt pathway. Lysate proteins binding to the fusion proteins can be resolved by SDS-PAGE, isolated and identified by, for example protein sequencing or mass spectroscopy, as is known in the art. See, e.g., PROTEIN PURIFICATION APPLICATIONS: A PRACTICAL APPROACH (Simon Roe, ed., 2^{nd} ed. Oxford Univ. Press, 2001) and “Guide to Protein Purification” in Meth. Enzymology vol. 182 (Academic Press, 1997).

The activity of Norrin, a Norrin mimetic, a Norrin interacting protein (e.g., Norrin agonist or Norrin antagonist), or a complex of Norrin with LRP5/LRP6/HBM and/or a Kremen protein or Dkk protein may be affected by compounds which modulate the interaction between Norrin and a Norrin interacting protein, and/or Norrin and LRP5/LRP6/HBM, Norrin and/or Frizzled4, a Dkk protein, or a Kremen protein. Provided herein are methods and research tools for the discovery and characterization of these compounds. The interaction between Norrin or a Norrin mimetic and a Norrin/Frizzled4 interacting protein and/or Norrin and LRP5/6/HBM, and Norrin/Dkk, and Norrin/Kremen may be monitored in vivo and in vitro. Similar assays can also be used for assessing Norrin agonists and antagonists. Compounds which modulate the stability of a Norrin/Fz4 complex are potential therapeutic compounds.

Example in vitro methods include: binding LRP5/6/HBM, Norrin/Fz4, or a Norrin/Fz4 interacting protein to a sensor chip designed for an instrument such are made by Biacore (Uppsala, Sweden) for the performance of a plasmon resonance spectroscopy observation. For example, using this method, a chip with one of Norrin/Fz4, a Norrin/Fz4 interacting protein, or LRP5/LRP6 can be first exposed to the other under conditions which permit them to form a complex. A test compound is then introduced, and the output signal of the instrument provides an indication of any effect exerted by the test compound. By this method, compounds may be rapidly screened. This method can be used for any Norrin/Fz combination with LRP5, LRP6, HBM, any Dkk, any Kremen, any Wnt, and any combination thereof.

Another, in vitro, method is exemplified by the SAR-by-NMR methods (Shuker et al., 1996 Science 274: 1531-4). For example, a Norrin/Fz4 binding domain and/or LRP5/LRP6/HBM LBD can be expressed and purified as ¹⁵N-labeled protein by expression in labeled media. The labeled protein(s) are allowed to form the complex in solution in a nuclear magnetic resonance (NMR) sample tube. The heteronuclear correlation spectrum in the presence and absence of a test compound provides data at the level of individual residues with regard to interactions with the test compound and changes at the protein-protein interface of the complex. This method can be used with any Norrin/Frizzled4 combination with LRP5, LRP6, HBM, any Dkk, any Kremen, any Wnt, and any combination thereof.

One of skill in the art knows of many other protocols, e.g., affinity capillary electrophoresis (Okun et al., 2001 J. Biol. Chem. 276: 1057-1062), fluorescence spectroscopy, electron paramagnetic resonance, etc., which can also be used to monitor the modulation of a complex and/or measure binding affinities for complex formation in the presence and absence of a test agent for any of the above listed combination of proteins or biologically active fragments thereof.

Protocols for monitoring the modulation of a Norrin/Frizzled4 interaction, a Norrin/LRP5/Frizzled4 interaction, or a Norrin mimetic's interaction with any one or more of LRP5, LRP6, HBM, Kremen 1, Kremen2, any Dkk, and any Wnt can be performed using a yeast hybrid protocol. The yeast two- or more hybrid method may be used to monitor the modulation of a complex by monitoring the expression of genes activated by the formation of a complex of fusion proteins of Norrin/Frizzled4 and/or any of the above-listed other proteins. If using LRP5, LRP6, or HBM, then the complete protein can be used or the ligand binding domains (LBDs) or portions of the beta propeller containing the YWTD repeats. Nucleic acids according to the invention which encode the interacting Norrin and Frizzled4 or Norrin and LRP5/LRP6/HBM LBD domains are incorporated into bait and prey plasmids. The yeast two hybrid (Y2H) method or yeast hybrid method for three or more proteins is performed in the presence of one or more test compounds. The modulation of the complex is observed by a change in expression of the complex activated gene. It will be appreciated by one skilled in the art that test compounds can be added to the assay directly or, in the case of proteins, can be co-expressed in the yeast with the bait and prey compounds. Similarly, fusion proteins of Norrin and Norrin interacting proteins can also be used in an Y2H screen to identify other proteins which modulate the Norrin/Frizzled4 complex (such as Dkk, Kremen, other negative regulators, and positive regulators). Yeast hybrid technologies are known in the art. See for example, Li ZHU AND GREGORY J. HANNON, YEAST HYBRID TECHNOLOGIES (2000).

Assay protocols such as these may be used in methods to screen for compounds, drugs, treatments which modulate the Norrin/Frizzled4 complex, whether such modulation occurs by competitive binding, acting as a Norrin mimetic, or by altering the structure of the Norrin/Frizzled4 complex, or by stabilizing or destabilizing the protein-protein interface. It may be anticipated that peptide aptamers may competitively bind, although induction of an altered binding site structure by steric effects is also possible. As used herein, a biological or pathological process modulated by Norrin/Frizzled4 and the Norrin/Fz4/LRP5 complex may include binding of Norrin to Frizzled4, or to a protein that interacts with the Norrin/Frizzled4/LRP5 complex, or prevents Dkk and/or Kremen down regulation of the Norrin/Frizzled4 complex. This can include compounds that interact with the Norrin or modulate synthesis of the proteins involved with the complex as well as Norrin mimetics.

Further bone-related markers may be observed such as alkaline phosphatase activity, osteocalcin production, or mineralization in addition to other bone related factors that can be assessed in conjunction with the biochemical analysis of modulation of the Norrin/Frizzled4/LRP5 complex, as discussed herein.

Pathological processes refer to a category of biological processes that produce a deleterious effect. For example, expression or up-regulation of expression of LRP5 or LRP6 and/or Dkk and/or a Dkk interacting protein may be associated with certain diseases or pathological conditions. As used herein, an agent is said to modulate a pathological process when the agent alters the process from its base level in the subject to a statistically significant level. For example, the agent may reduce the degree or severity of the process mediated by that protein in the subject to which the agent was administered. For instance, a disease or pathological condition may be prevented, or disease progression modulated by the administration of agents which reduce or modulate in some way the expression or at least one activity of a protein of the invention.

As Frizzled4/Norrin and LRP5/LRP6 (as well as Kremen, Dkk, and Wnt) are involved directly and/or indirectly in bone mass modulation, one embodiment of this invention is to use Norrin/Frizzled4 complex and Norrin/Frizzled4 complex ligands as a method of diagnosing a bone condition or disease. Certain markers are associated with specific Wnt signaling conditions (e.g., TCF/LEF activation). Diagnostic tests for bone conditions may include the steps of testing a sample or an extract thereof for the presence of Dkk or Dkk interacting protein nucleic acids (i.e., DNA or RNA), oligomers or fragments thereof or protein products of TCF/LEF regulated expression. For example, standard in situ hybridization or other imaging techniques can be utilized to observe products of Wnt signaling.

Also discussed herein are methods and materials for modulating bone development or bone loss conditions. Inhibition of bone loss may be achieved by inhibiting or modulating changes in the Norrin/Frizzled4 complex and thereby the Wnt signaling pathway. For example, absence of Norrin activity or increased Dkk1 activity may be associated with low bone mass. Increased activity Norrin and Frizzled4 may be associated with high bone mass. Therefore, modulation of Norrin/Frizzled4 activity will in turn modulate bone mass. Modulation of a Dkk's interaction with the Norrin/Frizzled4 complex via agonists and antagonists is one embodiment of a method to regulate bone development.

The agents of the present invention can be provided alone, or in combination with other agents that modulate a particular pathological process. As used herein, two agents are said to be administered in combination when the two agents are administered simultaneously or are administered independently in a fashion such that the agents will act at the same time.

The agents of the present invention can be administered to a non-human test animal for example via parenteral, subcutaneous (s.c.), intravenous (i.v.), intramuscular (i.m.), intraperitoneal (i.p.), transdermal or buccal routes. Alternatively, or concurrently, administration may be by the oral route. The dosage administered will be dependent upon the age, health, and weight of the recipient, kind of concurrent treatment, if any, frequency of treatment, and the nature of the effect desired.

The present invention further provides compositions containing one or more agents that modulate expression or at least one activity of Norrin or the Norrin/Frizzled4 complex or which act as a Norrin mimetic. While individual needs vary, determination of optimal ranges of effective amounts of each component is within the skill of the art. Typical dosages of the active agent, which include a Norrin mimetic or an agent that mediates Norrin, a Norrin interacting protein, or a ligand of the Norrin/Frizzled4 complex (or Norrin/Frizzled4/LRP5 complex, which is also contemplated throughout wherein Norrin/Frizzled4 complexes are discussed), may comprise from about 0.0001 to about 50 mg/kg body weight. The preferred dosages may comprise from about 0.001 to about 50 mg/kg body weight. The most preferred dosages may comprise from about 0.1 to about 1 mg/kg body weight. In an average human of 70 kg, the range would be from about 7 μg to about 3.5 g, with a preferred range of about 0.5 mg to about 5 mg (and for example any 0.1 mg value within this range).

In addition to the pharmacologically active agent, the compositions of the present invention may contain suitable pharmaceutically acceptable carriers comprising excipients, carriers, and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils (e.g., vegetable oils such as sesame oil), or synthetic fatty acid esters (e.g., ethyl oleate or triglycerides). Aqueous injection suspensions may contain substances which increase the viscosity of the suspension and include but are not limited to sodium carboxymethyl cellulose, sorbitol and/or dextran. Optionally, the suspension may also contain stabilizers. Liposomes and other non-viral vectors can also be used to encapsulate the agent for delivery into the cell.

The pharmaceutical formulation for systemic administration according to the invention may be formulated for enteral, parenteral, or topical (top) administration. Indeed, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

Potentially, any compound which binds and thereby modulates Norrin, a Norrin mimetic, a Norrin ligand, or the Norrin/Frizzled4 complex may be a therapeutic compound. In one embodiment of the invention, a peptide or nucleic acid aptamer according to the invention is used in a therapeutic composition. Such compositions may comprise an aptamer, or a Norrin/Frizzled4 fragment, unmodified or modified. In another embodiment, the therapeutic compound comprises a Norrin- or Norrin/Frizzled4-complex-interacting protein, or biologically active fragment thereof.

Nucleic acid aptamers have been used in compositions for example by chemical bonding to a carrier molecule such as polyethylene glycol (PEG), which may facilitate uptake or stabilize the aptamer. A di-alkylglycerol moiety attached to an RNA can be used to embed the aptamer in liposomes, thus stabilizing the compound. Incorporating chemical substitutions (i.e., changing the 2′-OH group of ribose to a 2′-NH in RNA confers ribonuclease resistance) and capping, etc. can prevent breakdown. Several such techniques are discussed for RNA aptamers in Brody and Gold, 2000 Rev. Mol. Biol. 74: 3-13.

Peptide aptamers may be used in therapeutic applications by the introduction of an expression vector directing aptamer expression into the affected tissue such as for example by retroviral delivery, by encapsulating the DNA in a delivery complex or simple by naked DNA injection. Or, the aptamer itself or a synthetic analog may be used directly as a drug. Encapsulation in polymers and lipids may assist in delivery. The use of peptide aptamers as therapeutic and diagnostic agents is reviewed by Hoppe-Syler and Butz, 2000 J. Mol. Med. 78: 426-430.

In another aspect, the structure of a constrained peptide aptamer of the invention may be determined such as by NMR or X-ray crystallography (Cavanagh et al., PROTEIN NMR SPECTROSCOPY: PRINCIPLES AND PRACTICE, Academic Press, 1996; Drenth, PRINCIPLES OF PROTEIN X-RAY CRYSTALLOGRAPHY, Springer Verlag, 1999). Preferably, the structure can be determined in complex with the target protein. A small molecule analog is then designed according to the positions of functional elements of the 3D structure of the aptamer. (GUIDEBOOK ON MOLECULAR MODELING IN DRUG DESIGN, Cohen, Ed., Academic Press, 1996; MOLECULAR MODELING AND DRUG DESIGN (TOPICS IN MOLECULAR AND STRUCTURAL BIOLOGY), Vinter and Gardner Eds., CRC Press, 1994). Methods are provided herein for the identifying and designing effective and specific drugs that modulate the activity of Norrin, act a Norrin mimetics, Norrin interacting proteins, Norrin/Frizzled4 interacting proteins, and the Norrin/Frizzled4 complex. Small molecule mimetics of the peptide aptamers are also encompassed within the scope.

2.1 Cell-Based Norrin Functional Reporter Assay

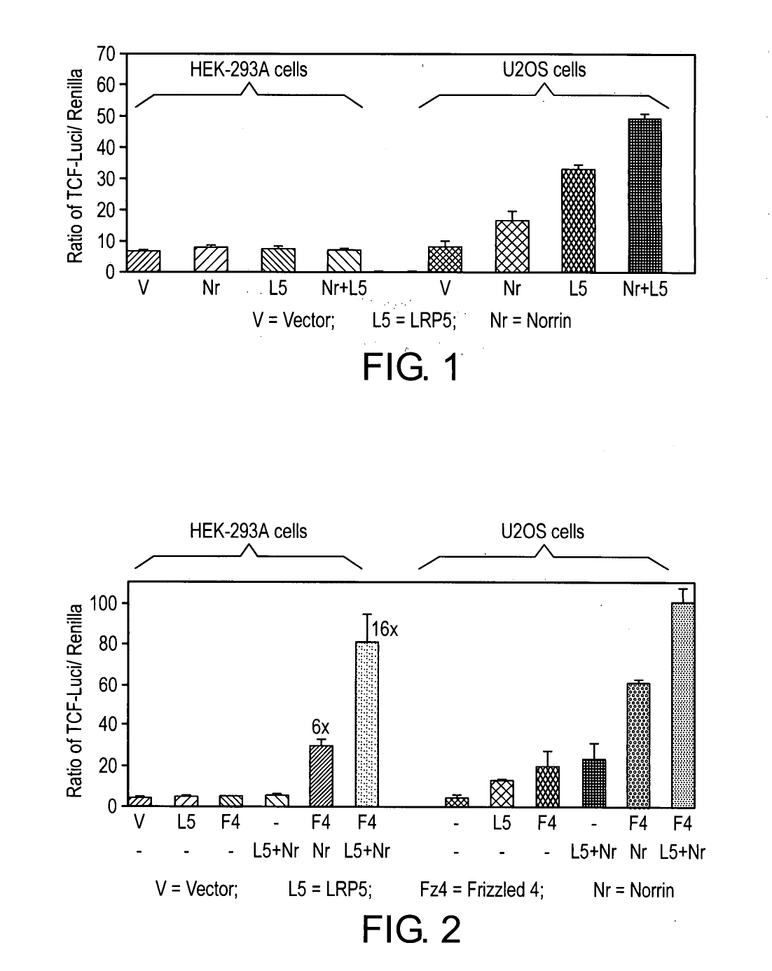

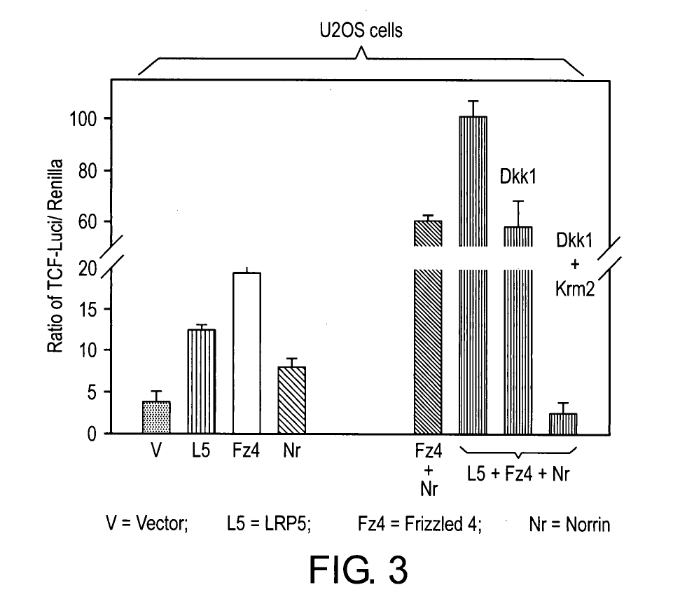

The TCF-reporter assays described in the examples below can be developed into screening assays either to identify Norrin mimetics (FIGS. 1 and 2) or to identify antagonists of Norrin-signal inhibitors, such as antagonists of Dkk1/Kremen2 (FIG. 3). In both types of assays when preformed in bone or non-bone cell types, the active molecules would enhance the TCF-luciferase signals or other suitable signal method.

2.2 Norrin/LRP5/LRP6 and DKK/LRP5/LRP6/Kremen Assays

Another method that can be used to screen for reagents that modulate Norrin's interaction with Frizzled4/LRP5/LRP6 is via an enzyme linked immunosorbent assay (ELISA) assay. Two possible permutations of this assay are exemplified, but others can also be utilized. For example, LRP5 can be immobilized to a solid surface, such as a tissue culture plate well. One skilled in the art would recognize that other supports, such as but not limited to a nylon or nitrocellulose membrane, a silicon chip, a glass slide, beads, etc. can be substituted and utilized. One manner of doing this can be to have the form of LRP5/LRP6/Fz4 as a fusion protein, wherein the extracellular domain of LRP5/LRP6/Fz4 is fused to the Fc portion of a human IgG or other IgG. The LRP5/LRP6/Fz4-Fc fusion protein can be produced in Chinese hamster ovary (CHO) cell (or another suitable cell line), wherein the fusion proteins are extracted from the cell lines or the media. The isolated LRP5/6/Fz4-Fc fusion protein can be immobilized on the solid surface via anti-human Fc antibody or by Protein-A or Protein G-coated plates, for example. The substrate can then be washed to remove any non-bound protein. Conditioned media containing secreted Norrin protein or secreted Norrin-epitope tagged protein (or purified Norrin, purified Norrin-epitope tagged protein, Norrin mimetic, or fragment containing a biologically active portion of Norrin involved in bone modulation) can be incubated in the wells or containers. Alternatively, a test reagent can be incubated with the fixed fusion protein in order to screen for Norrin mimetics. The binding of Norrin or Norrin mimetic to LRP5/LRP6/Fz4 can be assessed using antibodies to either Norrin or to an epitope tag. For example, a Norrin-V5 epitope tagged protein or fragment thereof can be detected with anti-V5 antibody. This assay system can then be used for example to identify Norrin mimetics, Norrin agonists, and immunoglobulins that bind in a manner like Norrin to the LRP5/LRP6/Fz4 fusion protein. Assays can be, for example, in the form of a competitive assay, tagged test reagents, and the like. These assay systems can also be utilized with HBM. The assay can also be modified to have washings that include a Dkk, a Kremen, and/or a Wnt protein or biologically active fragment thereof, when screening for test agents that modulate the interaction and formation of complexes between these proteins and polypeptides.

Alternatively, the Norrin protein or a biologically active fragment thereof (all references to Norrin protein assumes that a biologically active fragment can also be used) or a Norrin mimetic, which is involved in bone modulation could be directly fused to a detection marker, such as alkaline phosphatase. Here the detection of the Norrin-LRP5/LRP6/Fz4 interaction can be directly investigated without subsequent antibody-based experiments. The bound Norrin or Norrin mimetic is detected in an alkaline phosphatase assay or other detection assay. If the Norrin-alkaline phosphatase fusion protein is bound to the immobilized LRP5/LRP6/Fz4, alkaline phosphatase activity would be detected in a calorimetric, radioactive, or fluorescent readout. As a result, one can assay the ability of small molecule compounds to alter the binding of Norrin to LRP5/LRP6/Fz4 using this system or whether the test reagent is a Norrin mimetic or Norrin agonist. For example, compounds, when added with Norrin (or epitope-tagged Norrin) to each well of the plate, can be scored for their ability to modulate the interaction between Norrin and LRP5/LRP6/Fz4 based on the signal intensity of bound Norrin present in the well after a suitable incubation time and washing. The assay can be calibrated by doing competition experiments with unlabeled Norrin or with a second type of epitope-tagged Norrin. Any molecule that is able to modulate (e.g., enhance) the Norrin-LRP5/LRP6/Fz4 interaction may be a suitable therapeutic candidate, more preferably an osteogenic therapeutic candidate or a candidate capable of modulating a lipid (e.g., ApoE, LDL, HDL, VLDL, triglyceride, cholesterol). Such molecules include small chemical compounds, peptides, and immunoglobulins; (antibodies, antibody fragments) all can be examined using such an assay system.

2.3 Norrin-LRP5/6/Fz4 Homogenous Assay

Another method to investigate modulation of protein-protein interactions is via Fluorescence Resonance Energy Transfer (FRET). FRET is a quantum mechanical process, where a fluorescent molecule, the donor, transfers energy to an acceptor chromophore molecule which is in close proximity. Similarly an Amplified Luminescent Proximity Homogenous Assay (ALPHA screen) also can be used to evaluate Norrin-LRP5/6 or Fz4 interaction domains and function of Norrin mimetics in the Fz4/LRP5 complex. Such systems have been successfully used in the literature to characterize the intermolecular interactions between LRP5 and Axin (see, e.g., Maio et al., Molec. Cell Biol. 7: 801-9). There are many different fluorescent tags available for such studies and there are several ways to fluorescently tag the proteins of interest. For example, CFP (i.e., cyan fluorescent protein) and YFP (i.e., yellow fluorescent protein) can be used as donor and acceptor, respectively. Fusion proteins, with a donor and an acceptor, can be engineered, expressed, and purified or conjugated to specific donor and acceptor beads.

For instance, in FRET type assays, purified Norrin proteins, or biologically active polypeptides thereof, or agents being screened as Norrin mimetics can be fused to CFP (or another fluorescent protein), and purified LRP5/6/Fz4 protein or biologically active polypeptides thereof (e.g., LBD, or beta propeller containing domain), fused to YFP can be generated and purified using standard approaches. If Norrin-CFP and LRP5/Fz4-YFP are in close proximity, the transfer of energy from CFP to YFP will result in a reduction of CFP emission and an increase in YFP emission. Energy is supplied with an excitation wavelength of 450 nm, and the energy transfer is recorded at emission wavelengths of 480 nm and 570 nm. The ratio of YFP emission to CFP emission provides a gauge for changes in the interaction between Norrin (or Norrin mimetic) and LRP5/Fz4. This system is amenable for screening small molecule compounds that may alter the Norrin-LRP5/Fz4 protein-protein interaction and activity in the Wnt cascade. Compounds that enhance or disrupt the interaction would be identified by an increase or decrease respectively in the ratio of YFP emission to CFP emission. Such compounds that modulate the LRP5/Fz4 interaction in the same fashion as Norrin would then be considered candidate Norrin mimetic molecules. Agents would also be screened for those which enhance Norrin-like activity. These assay systems can be further modified with different fluorescent proteins to include Kremen, Dkk, and/or Wnt in various combinations. Further characterization of the compounds can be done using the TCF-luciferase or Xenopus embryo assays to elucidate the effects of the compounds on functional Norrin signaling.

2.4 Yeast Hybrid Assays

The two-hybrid, three-hybrid or other yeast hybrid system is extremely useful for studying protein:protein interactions. See, e.g., Chien et al., 1991 Proc. Nat'l Acad. Sci. USA 88: 9578-82; Fields et al., 1994 Trends Genetics 10: 286-92; Harper et al., 1993 Cell 75: 805-16; Vojtek et al., 1993 Cell 74: 205-14; Luban et al., 1993 Cell 73: 1067-78; Li et al., 1993 FASEB J 7: 957-63; Zang et al., 1993 Nature 364: 308-13; Golemis et al., 1992 Mol. Cell. Biol. 12: 3006-14; Sato et al., 1994 Proc. Nat'l. Acad. Sci. USA 91: 9238-42; Coghlan et al., 1995 Science 267: 108-111; Kalpana et al., 1994 Science 266: 2002-6; Helps et al., 1994 FEBS Lett. 340: 93-8; Yeung et al., 1994 Genes & Devel. 8: 2087-9; Durfee et al., 1993 Genes & Devel. 7: 555-569; Paetkau et al., 1994 Genes & Devel. 8: 2035-45; Spaargaren et al., 1994 Proc. Nat'l. Acad. Sci. USA 91: 12609-13; Ye et al., 1994 Proc. Nat'l Acad. Sci. USA 91: 12629-33; and U.S. Pat. Nos. 5,989,808; 6,251,602; and 6,284,519.

Variations of the system are available for screening yeast phagemid (see, e.g., Harper, CELLULAR INTERACTIONS AND DEVELOPMENT: A PRACTICAL APPROACH, 153-179 (1993); and Elledge et al., 1991 Proc. Nat'l Acad. Sci. USA 88: 1731-5), or plasmid (Bartel, 1993 Cell 14: 920-4); Finley et al., 1994 Proc. Nat'l Acad. Sci. USA 91: 12980-4) cDNA libraries to clone interacting proteins, as well as for studying known protein pairs.

The success of the two-hybrid system relies upon the fact that the DNA binding and polymerase activation domains of many transcription factors, such as GAL4, can be separated and then rejoined to restore functionality (Morin et al., 1993 Nuc. Acids Res. 21: 2157-63). While these examples describe two-hybrid screens in the yeast system, it is understood that a two-hybrid screen may be conducted in other systems such as mammalian cell lines. The invention is therefore not limited to the use of a yeast two-hybrid system, but encompasses such alternative systems.

Yeast strains with integrated copies of various reporter gene cassettes, such as for example GAL→LacZ, GAL→HIS3 or GAL→URA3 (Bartel, IN CELLULAR INTERACTIONS AND DEVELOPMENT: A PRACTICAL APPROACH, 153-179 (1993); Harper et al., 1993 Cell 75: 805-16; Fields et al., 1994 Trends Genetics 10: 286-92) are co-transformed with two plasmids, each expressing a different fusion protein. One plasmid encodes a fusion between protein “X” and the DNA binding domain of, for example, the GAL4 yeast transcription activator (Brent et al., 1985 Cell 43: 729-36; Ma et al., 1987 Cell 48: 847-53; Keegan et al., 1986 Science 231: 699-704), while the other plasmid encodes a fusion between protein “Y” and the RNA polymerase activation domain of GAL4 (Keegan et al., 1986). The plasmids are transformed into a strain of the yeast that contains a reporter gene, such as lacZ, whose regulatory region contains GAL4 binding sites. If proteins X and Y interact, they reconstitute a functional GAL4 transcription activator protein by bringing the two GAL4 components into sufficient proximity to activate transcription. It is well understood that the role of bait and prey proteins may be alternatively switched and thus the embodiments of this invention contemplate and encompass both alternative arrangements.

Either hybrid protein alone must be unable to activate transcription of the reporter gene. The DNA-binding domain hybrid must be unable to activate transcription, because it does not provide an activation function; and the activation domain hybrid must be unable to activate transcription, because it cannot localize to the GAL4 binding sites. Interaction of the two test proteins reconstitutes the function of GAL4 and results in expression of the reporter gene. The reporter gene cassettes consist of minimal promoters that contain the GAL4 DNA recognition site (Johnson et al., 1984 Mol. Cell. Biol. 4: 1440-8; Lorch et al., 1984 J. Mol. Biol. 186: 821-824) cloned 5′ to their TATA box. Transcription activation is scored by measuring either the expression of β-galactosidase (or other reporter) or the growth of the transformants on minimal medium lacking the specific nutrient that permits auxotrophic selection for the transcription product, e.g., URA3 (uracil selection) or HIS3 (histidine selection). See, e.g., Bartel, 1993; Durfee et al., 1993 Genes & Devel. 7: 555-569; Fields et al., 1994 Trends Genet. 10: 286-292; and U.S. Pat. No. 5,283,173.

Generally, these methods include two proteins to be tested for interaction which are expressed as hybrids in the nucleus of a yeast cell. One of the proteins is fused to the DNA-binding domain (DBD) of a transcription factor, and the other is fused to a transcription activation domain (AD). If the proteins interact, they reconstitute a functional transcription factor that activates one or more reporter genes that contain binding sites for the DBD. Exemplary two-hybrid assays are Norrin, Norrin/Frizzled4, or Frizzled4/LRP5 fusions.

3. In vivo Methods of Assaying Agents

In addition to the in vitro methods identified herein, the methods and materials can further include use of animals to study the effect of test agents screened and identified by in vitro analysis. For example, transgenic animals wherein one (or more) of Norrin, Kremen (Kremen 1 and/or 2), Dkk (Dkk1, Dkk2, Dkk3, and/or Dkk4), LRP5, LRP6, HBM, Wnt (Wnt1 to Wnt19), and Frizzled4 genes are introduced as cDNAs. Examples of LRP5 and HBM transgenic animals can be found in International PCT Application No. PCT/US02/14876 and U.S. application Ser. No. 10/680,287. The subject matter of these applications is incorporated herein by reference in their entirety for all purposes.

Thus, in one aspect, after the steps of screening the test agent against any of the transfected cell lines discussed above and/or after agents have been tested to see if they bind to any of Dkk, Norrin, Frizzled4, LRP5, LRP6, HBM, Wnt, and/or Kremen, these test agents can also be assessed in vivo. Adding the step of testing reagents in vivo adds a validation step to the tests obtained by any of the means discussed in Section 2 supra. Reagents can be administered to the animals via any means of administration suitable for the compound, e.g., oral, intravenous, intramuscular, intraperitoneal, cutaneous, and the like. Administration may depend on the formulation of the test compound. For example, small inhibitory RNAs (siRNAs) and immunoglobulins may get administered intravenously rather than orally. Small chemical compounds may be administered orally or intravenously. Amounts of the test compound would be administered based on a weight basis for the animal.

Animals could also be utilized to test bioavailability and degradation products of the compounds.

Animals would be administered the test compounds over a period of days, weeks, or months. Administration can be daily, weekly, bimonthly, monthly and the like. Animals can have the agent administered alone or in conjunction with exercise, which causes strain on the bones of the animal. Discussion of how strain can be placed on the animals' bones is described in International PCT Application No. PCT/US2004/17951. The contents of this application are incorporated herein by reference in its entirety for all purposes.

For example, the pDXA can be measured in wild-type and transgenic animals that are administered various dosages of agents. For example, wild-type and transgenic mice are anesthetized, weighed and whole-body X-ray scans of the skeleton generated using the LUNAR small animal PIXImus device. Scans can be performed when the mice are weaned (i.e., at 3 weeks of age) and repeated at 2 week intervals. Wild-type animals can be scanned at 3, 5, 7, 9, 11, 13, 15, 17, 19, 21, 23, 27, and 29 weeks. Scanning of transgenic animals can be performed for periods up to 17 weeks. Scans can be analyzed for BMD (bone mineral density), BMC (bone mineral content), TTM (total tissue mass), and percent (%) fat for various body regions.

Additionally or alternatively, faxitron radiographs of the above animals can be obtained. For example, following pDXA scanning of anesthetized animals, an additional X-ray can be taken using a Faxitron device allowing measurement of bone size.

Additionally or alternatively, calcein labeling can be performed on the above animals. For example, animals can be dosed with calcein at 15 mg/kg animal weight on two consecutive occasions. The first dose can be given 9 days before the animal is euthanized, and the second dose given two days prior to animal euthanasia. Measurement of bone formation can then be determined.

Certain types of ex vivo analysis of the above animals can also optionally be performed. For example, RNA isolation can be done from tissue, pQCT and microCT (ACT), histology, bending strength analysis, compressive strength analysis of vertebra, and serum analysis can be performed. For example, RNA can be isolated from tibia and other tissues using TRIzo17 to determine mRNA expression. pQCT analysis of any of the above animals can be performed by obtaining a femur and cleaning it of soft tissue. The femur can then be stored in 70% ethanol for determination of total and trabecular density of the distal metaphysis and cortical density of the mid-shaft then determined.

Analysis of the animal's femur can also be used to determine trabecular indices of the distal metaphysis.

Optionally, histological analysis can be performed on any of the above animals. For example, the femur of a mouse can be used to determine bone area and static and dynamic parameters of the distal metaphysis. Alternatively or additionally, immunohistochemistry can be performed (e.g., in situ hybridization of osteogenic markers and TUNEL staining of cells undergoing apoptosis).

Any of the above animals can also have their bones examined for bending strength or compressive strength of vertebra. For bending strength, the animal's femur (or other suitable bone) can be cleaned of soft tissue and stored at about −20° C. prior to analysis of 3-point bending strength of the mid-shaft. Compressive strength can be measured by removing the spine of, for example, a mouse from T10 to L6 or L7. Soft tissue is left on the spine which is then frozen at about −20° C. until analysis. Compressive strength is frequently measured at the L5 vertebra.

For purposes of lipid analysis, serum from animals can be assessed. For example, animals can be euthanized and serum prepared from the blood to measure total cholesterol, triglycerides, osteocalcin, and other biochemical surrogate markers.

Gene transcript and expression modulation can also be assessed on animals. For example, load on bones is known to impact the genes as follows:

TABLE 1
Effect of Load on Gene Expression in vivo
Comparing HBM TG and Non-TG Animals

| Gene | Pathway | Effect of Load on Gene Expression |
|---|---|---|
| ACP5 | HBM | Up-regulated equally in the males and |
| | | is more significantly induced in female |
| | | HBM-TG |
| Col1A1 | HBM | No significant change in either |
| Connexin 43 | Wnt | Up-regulated; More significant in HBM-TG |
| CTSK | HBM | Up-regulated in both animals equally |
| Cyclin D1 | Wnt | Up-regulated; More significant in HBM-TG |
| ENOS | Load Sensor | Up-regulated; More significant in HBM-TG |
| Frizzled 2 | Wnt | Up-regulated; More significant in HBM-TG |
| GADD45A | HBM | Down-regulated in both animals |
| IGF2 | HBM | Up-regulated in both male animals |
| IGFBP6 | HBM | Up-regulated; more significant in HBM-TG |
| IL-6 | Load Sensor | Up-regulated; More significant in HBM-TG |
| IL-8 | Stress & | Up-regulated; More significant in HBM-TG |
| | Osteoclast | |
| | Function | |
| LRP5 | — | No significant change in either |
| MK2 | Stress & | Up-regulated in non-TG animals only |
| | Osteoclast | |
| | Function | |
| OPG | Stress & | Up-regulated in HBM-TG animals only |
| | Osteoclast | |
| | Function | |
| Osteonectin | HBM | Up-regulated; More significant in HBM-TG |
| PTGS | Load Sensor | Up-regulated; More significant in HBM-TG |
| RANKL | Stress & | No significant change in either |
| | Osteoclast | |
| | Function | |
| SFRP1 | Wnt | Up-regulated; More significant in HBM-TG |
| SFRP4 | Wnt | Up-regulated; More significant in HBM-TG |
| TGFβ | HBM | No significant change in either |
| TIMP3 | HBM | No significant change in either |
| WISP2 | Wnt | Up-regulated; More significant in HBM-TG |
| Wnt10B | Wnt | Up-regulated; More significant in HBM-TG |

Any one or more of the above referenced genes, in any combination, can be assessed for changes in expression due to administration of a test agent, control agent, stress on the bone or bone cell, and so forth. The gene profile can be produced and then be assessed in conjunction with the affect the agent has on the Norrin-Frizzled4 or Frizzled4-LRP5 complex and its activity in the Wnt pathway. For example, the gene/protein profile obtained by the agent that modulated the Norrin-Frizzled4-LRP5 complex or a Norrin mimetic produces a profile like that observed either with the administration of a Dkk antagonist or Kremen antagonist or by stress on the bone or bone cell.

Bone load and comparing bone load to modulation by an agent can also be performed in vitro. For example, gravitational load can be used to induce stress on any of the bone cells discussed herein, and the profile of gene expression of any one or more of the genes or any combination of the following genes can be assessed as part of a bone stress profile. The bone stress profile can be assessed to see whether, for example, a Norrin mimetic induces an enhance bone stress profile or decreases the inhibition of Dkk and/or Kremen on the genes of a bone stress profile.

TABLE 2
Effect of Load on Gene Expression In vitro

| | | MC3T3 Cell |
|---|---|---|
| | | Response to |
| | | Gravitational |
| Gene | Gene type | Load |
| AP1B1 | Stress regulated gene | Up-regulated |
| AXIN | Wnt pathway component | Up-regulated |
| BMP1 | Observed to be induced by | Up-regulated |
| | iGSK-3 | |
| CBFB | Osteoblast function | Up-regulated |
| CCND1 | Wnt target gene | Up-regulated |
| CCND3 | Cell cycle | Up-regulated |
| CELSR2 | G-type receptor | Up-regulated |
| CHUK/IKK alpha | Facilitates β-catenin nuclear | Up-regulated |
| | translocation | |
| CK1 alpha | Wnt pathway component | Up-regulated |
| CKB | Kinase | Up-regulated |
| CRABP2 | Osteoblast differentiation | Up-regulated |
| CSF1R | Osteoclastogenesis | Up-regulated |
| CTGF | Growth factor | Up-regulated |
| DVL1 | Wnt signaling intermediate | Up-regulated |
| EPHB6 | Wnt target gene | Up-regulated |
| FOSL1 | Stress regulated gene | Up-regulated |
| GADD45B | Cell cycle | Up-regulated |
| GADD45G | Cell cycle | Up-regulated |
| GJA1 | Wnt target gene | Up-regulated |
| GJB3 | Wnt target gene | Up-regulated |
| HERPUD1 | Wnt target gene | Up-regulated |
| IGFBP6 | | Up-regulated |
| IL1R1 | IL-1 mediated signaling, | Up-regulated |
| | inflammation | |
| IL1RL1 | IL-1 mediated signaling, | Up-regulated |
| | Inflammation | |
| IL4RA | Inflammation | Up-regulated |
| ITGA5 | Integrin signaling | Up-regulated |
| JUN | Stress regulated gene | Up-regulated |
| JUND1 | Stress regulated gene | Up-regulated |
| LDLR | Lipoprotein receptor | Up-regulated |
| LOX | Lysyl oxidase | Up-regulated |
| MAPKAPK2 | Kinase in stress regulated | Up-regulated |
| | signaling | |
| MSX1 | Wnt target gene | Up-regulated |
| MYCS | Wnt target gene | Up-regulated |
| NCAM1 | Wnt target gene | Up-regulated |
| NFATC1 | Inflammation | Up-regulated |
| NFKB1 | Inflammation, proliferation | Up-regulated |
| PDGFA | Growth factor, osteoblast | Up-regulated |
| | development | |
| PRDC-PENDING | Cereberus like protein | Up-regulated |
| PTGS1 | Inflammation | Up-regulated |
| PTGS2 | Wnt target gene | Up-regulated |
| RAMP3 | Calcium signaling | Up-regulated |
| RUNX | Osteoblast function | Up-regulated |
| RUNX2/CBFA1 | Osteoblast function | Up-regulated |
| SDC1 | Proteoglycan required for Wnt | Up-regulated |
| | signaling | |
| SERPINE1 | Protease | Up-regulated |
| SPARCL1 | Osteoblast function | Up-regulated |
| STAT3 | Proliferation and cell growth | Up-regulated |
| TANK | Inflammation, NF-kB signaling | Up-regulated |
| TGFB1 | TGF beta signaling gene | Up-regulated |
| THBD | Endothelial cell function | Up-regulated |
| TIEG | TGF beta signaling gene | Up-regulated |
| TIMP1 | Matrix metalloproteinase | Up-regulated |
| TIMP3 | Matrix metalloproteinase | Up-regulated |
| TNFRSF11B/OPG | Wnt target gene | Up-regulated |
| TRAF3 | NF-kB signaling | Up-regulated |
| WISP1 | Wnt target gene | Up-regulated |

Determination of whether the administered test agents can induce a bone modulating effect can be assessed by X-ray for change of bone density or by animal sacrifice and examination of cortical bone as described in U.S. application Ser. No. 10/680,287 and International PCT Application No. PCT/US2004/17951, or any of the methods described herein or known in the art. The contents of these applications are incorporated herein by reference in their entirety for all purposes.

4. Methods of Studying Bone Loading In vitro

One aspect of the invention is the study of the effect of bone load in vitro and means by which the benefits of bone load (i.e., increased bone mineralization) can be enhanced. Studying bone load enhancement can be done both in vivo (as discussed above) and in vitro. Bone load enhancement can be first performed in vitro followed then with in vivo experiments, such as those discussed above.

Consequently, one aspect of the invention involves placing cells under conditions, which simulate load stimuli. There are several methods available for placing strain on cell cultures to mimic the bone load response observed in vivo. These methods include but are not limited to fluid shear, hydrostatic compression, uniaxial stretch, biaxial stretch, gravitational loading and load induced using a Flexercell®, or equivalent system.

4.1 Bone Load Stimuli

Preferred genes which are modulated by a bone load stimuli, such as those provided by any of the above methods, include but are not limited to SFRP1, connexin, WISP2 43, CCND1, Wnt10b, Jun, Fos, PTGS2 (COX-2), and eNOS. Additional genes that can be monitored for increases in their activity (e.g., increased mRNA transcripts and protein) as reflected in many of the Tables herein. At least six genes that have been shown to be consistently up-regulated in response to bone load (i.e., Jun, Fos, eNOS, SFRP1, COX-2 and Connexin 43) are also enhanced by the addition of an agent which activates the Wnt pathway. Other genes, such as Wnt2, are not enhanced by the addition of reagents that activated the Wnt pathway (e.g., GSK-3 inhibitors and Wnt 3A and its agonists, mimetics, and variants) and only respond to bone load. Thus, one aspect would include using such in vitro systems to study enhancement of the stress profile genes in response to, for examples, a Norrin mimetic, a Norrin agonist, a Frizzled4 mimetic, or a Frizzled4 mimetic.

4.1.1 Fluid Shear Stimulus

One method of inducing bone load is by fluid shear. Fluid shear can utilize a cone plate viscometer that generates continuous laminar shear by a stirring mechanism. Alternatively, a flow loop apparatus can produce such shear in a parallel flow culture chamber. The latter method and apparatus is exemplified by the Streamer system produced by Flexcell International Corporation. The flow loop apparatus also is known to produce a reproducible and consistent stimulus. The only drawbacks are that the end points are typically short-lived and whether these changes impact the function of differentiated osteoblasts (Basso et al., 2002 Bone 30(2): 347-51).

4.1.2 Hydrostatic Compression Stimulus

A second method of inducing bone load is use of hydrostatic compression. Hydrostatic compression can utilize compressed air to generate a continuous or intermittent force that is believed to localize the force specifically to regions where the cells interact with the extracellular matrix protein/adhesion proteins.

4.1.3 Uniaxial Stretch Stimulus

A third means of inducing bone load in vitro is use of a uniaxial stretch stimulus. The uniaxial stretch method utilizes stretch force in one direction. The method involves growing cells in a tissue culture on a treated strip of polystyrene film or other film, which is fixed to a flexible layer of silicone. The layer of silicone is further attached to two metal bars. The metal bars can be manipulated relative to each other using an electromagnet or some other moving means. This method does not create any fluid shear. The lack of fluid shear makes this method less preferred, because interstitial fluid flow may play a larger role in bone remodeling than mechanical stretch. Accordingly, this method may not fully mimic what occurs in vivo despite the reproducible and consistent stimulus produced (Basso et al., 2002 Bone 30(2): 347-51).

4.1.4 Biaxial Stretch Stimulus

Biaxial stretch is essentially the Flexercell® system discussed herein. This method uses a collagen coated silastic membrane upon which the cells are grown. The plates are then placed in a special tray, which is attached to a vacuum pump. The vacuum pump stretches and relaxes the membrane, by stretching or otherwise distorting the cell membrane. Additionally, any media or fluid movement will further add fluid shear.

4.1.5 Gravitational Load Stimulus

Gravitational loading is another method by which bone load can be induced in vitro. Essentially, force is placed on the cells causing the cells to flatten. For additional details, see for example, Hatton et al., 2003 J Bone & Min. Res. 18(1): 58-66; and Fitzgerald et al., 1996 Exp. Cell. Res. 228: 168-71. Specifically, the cells are grown on plates or cover slips and then are exposed to increasing G forces.

4.1.6 Flexercell® Stimulus

One preferred method for assessing reagent-based enhancement of the Wnt pathway and bone mineralization is using the Flexercell® system, a biaxial stretch stimulus. Briefly, bone cells (e.g., MC3T3 cells) are exposed to about 3,400 με. Loads of about 50 με to about 5,000 με (and any value in between) can be used as well for mechanical load stimuli. Any stimulus in this range mimics physiological bone load stimuli. Stimuli above 5,000 με result in pathophysiological loads, and therefore are not preferred. The cells also can be exposed to a Wnt pathway modulator (e.g., a GSK inhibitor) prior to exposure to biaxial stretch.

The genes up-regulated by the administration of the load alone or with a GSK-3 inhibitor include, but are not limited to COX-2, eNOS, connexin 43, Fos, Jun, WISP2, Wnt10b, Cyclin D1, and SFRP1. The expression profile obtained in vitro from the Flexercell® studies mimics the in vivo loading gene expression profile (i.e., RNA analysis performed on cells from HBM TG mice tibia wherein the mice were subjected to bone load using a four-point system). Thus, this mechanical load assay, or the use of other mechanical load means with the variety of cell lines disclosed herein, can be used to identify small molecules, peptides, immunoglobulins, and the like that modulate, and preferably activate, the canonical Wnt pathway and which mimic the HBM phenotype. A Norrin mimetic can produce the same response as Norrin or an enhanced response, like the enhanced response of the HBM variant of LRP5 of increased bone mass. Thus, using this system would be helpful for screening reagents that enhance the up-regulated genes of the stress profile in an HBM-like manner as well as acting in a manner equivalent to wild-type Norrin.

The in vitro methods of inducing mechanical stress stimuli on cells can also be V used to study cell proliferation and apoptosis, which is relevant to bone remodeling and the need for osteoblast and osteoclast proliferation and osteoclast resorption. For example, HBM and unaffected osteoblastic cells can be seeded into bioflex 6-well plates and cultured for 2-3 days in growth media containing 10% FBS until the cells are about 60% confluent. Twenty-four hours prior to mechanical loading, the media is replaced with 1 mL of basal media containing about 2 to about 4% FBS. The cells are then subjected to about 50 to about 5,000 με of load for about 1 to about 5 hours. The cells can be further studied for reagents that are Norrin mimetics or which are agonists of the LRP5/Norrin/Frizzled4 complex (e.g., Norrin agonists, Frizzled4 agonists, or LRP5 agonists) as well as antagonists thereof.

Following load, the cells are cultured for an additional period of time. Subsequently, cell number and proliferation can be assessed using a number of commercial assays or assays known in the art, including but not limited to [³H]-thymidine incorporation, 5-bromo-2′-deoxyuridine (BrdU) incorporation, 3-(4,5 dimethylthiazol-2-yl)-5-(3-carboxymethoxyphenyl)-2-(4-sulfophenyl)-2H-trazolium salts (MTS) assay, TUNEL assay (i.e., terminal deoxynucleotidyltransferase dUTP nick end labeling) or Annexin V assay.

The following genes can be analyzed with regard to the profile. In another embodiment, Wnt antagonists can be screened or used to treat individuals wherein bone demineralization (e.g., osteopetrosis) is needed. Wnt antagonists include but are not limited to Dkk1 antagonists, and Kremen antagonists. Norrin agonists, and Norrin mimetics along with Frizzled4 agonists and mimetics, Wnt agonists and mimetics, and LRP5 and LRP6 agonists and mimetics can also be assessed under this system.

TABLE 3
Genes for Development of High Bone Mass
Microarray or Protein/Antibody Array

| | | WHERE |
|---|---|---|
| GENE | DESCRIPTION | EXPRESSED |
| ACP5 | acid phosphatase 5, tartrate | Bone and |
| | resistant | colon cancer |
| CCND1 | cyclin D1 (PRAD1: parathyroid | HBM Bone |
| | adenomatosis 1) | |
| CNK1 | v-erb-b2 erythroblastic leukemia | Bone and |
| | viral oncogene homolog 3 (avian) | colon cancer |
| COL1A1 | collagen, type I, alpha 1 | HBM Bone |
| COL6A3 | collagen, type VI, alpha 3 | HBM Bone |
| CTGF | connective tissue growth factor | HBM Bone |
| CTSK | cathepsin K (pycnodysostosis) | HBM Bone |
| CX3CR1 | chemokine (C-X3-C) receptor 1 | Inflammation |
| | | in bone |
| DELTEX | deltex homolog 2 (Drosophila), | Bone and |
| | EphB2 | colon cancer |
| EPHB2 | connector enhancer of KSR-like | Bone and |
| | (Drosophila kinase suppressor | colon cancer |
| | of ras) | |
| ERBB3 | GRO1 oncogene (melanoma growth | Bone and |
| | stimulating activity, alpha) | colon cancer |
| FAP | fibroblast activation protein, | Bone and |
| | alpha | colon cancer |
| FBLN1 | fibulin 1 | HBM Bone |
| FGF-2 | fibroblast growth factor 2 | Inflammation |
| | (basic) | in bone |
| FGF-7 | fibroblast growth factor 7 | Inflammation |
| | (keratinocyte growth factor) | in bone |
| FOS | fos FBJ murine osteosarcoma | Bone and |
| | viral oncogene homolog | colon |
| | | cancer/load |
| | | sensing gene |
| FZD2 | frizzled (Drosophila) homolog 2 | HBM Bone |
| GADD45A | growth arrest and DNA-damage- | HBM Bone |
| | inducible, alpha | |
| GAS6 | growth arrest-specific 6 | HBM Bone |
| GJA1 | gap junction protein, alpha 1, | HBM Bone |
| | 43 kD (connexin 43) | |
| IGF2 | insulin-like growth factor 2 | Inflammation |
| | (somatomedin A) | in bone |
| IGF2R | insulin-like growth factor 2 | Inflammation |
| | receptor | in bone |
| IGFBP6 | insulin-like growth factor | HBM Bone |
| | binding protein 6 | |
| IL-6 | interleukin 6 (interferon, | Inflammation |
| | beta 2) | in bone |
| ITGB5 | integrin, beta 5 | HBM Bone |
| ITGBL1 | integrin, beta-like 1 (with | HBM Bone |
| | EGF-like repeat domains) | |
| JUN | jun avian sarcoma virus 17 | Bone and |
| | oncogene homolog | colon |
| | | cancer/load |
| | | sensing gene |
| LOX | lysyl oxidase | HBM Bone |
| LRP5 | low density lipoprotein receptor- | HBM Bone |
| | related protein 5 | |
| LRP6 | low density lipoprotein receptor- | HBM Bone |
| | related protein 6 | |
| LSP1 | lymphocyte-specific protein 1 | Inflammation |
| | | in bone |
| MAPKAPK2 | mitogen-activated protein kinase- | Osteoclast |
| | activated protein kinase 2 | activity |
| MCC | mutated in colorectal cancers | Bone and |
| | | colon cancer |
| MET | met proto-oncogene (hepatocyte | HBM Bone |
| | growth factor receptor) | |
| MYBL1 | v-myb myeloblastosis viral | HBM Bone |
| | oncogene homolog (avian)-like 1 | |
| MYC | v-myc avian myelocytomatosis | Bone and |
| | viral oncogene homolog | colon cancer |
| eNOS | nitric oxide synthase 3 | Load |
| | (endothelial cell) | responsive |
| | | genes |
| OSMR | oncostatin M receptor | HBM Bone |
| PDGFRA | platelet-derived growth factor | HBM Bone |
| | receptor, alpha polypeptide | |
| PTGS2/ | prostaglandin-endoperoxide | Load |
| COX-2 | synthase 2 (prostaglandin G/H | responsive |
| | synthase and cyclooxygenase) | genes |
| SFRP1 | secreted frizzled-related | HBM Bone |
| | protein 1 | |
| SFRP4 | secreted frizzled-related | HBM Bone |
| | protein 4 | |
| SPARC | sparc/osteonectin, cwcv and | Inflammation |
| | kazal-like domains proteoglycan | in bone |
| | (testican) | |
| STAT1 | signal transducer and activator | Inflammation |
| | of transcription 1, 91 kD | in bone |
| TGFBR2 | transforming growth factor, beta | Inflammation |
| | receptor II (70-80 kD) | in bone |
| THBS1 | thrombospondin 1 | HBM Bone |
| TIMP2 | tissue inhibitor of | HBM Bone |
| | metalloproteinase 2 | |
| TIMP3 | tissue inhibitor of | HBM Bone |
| | metalloproteinase 3 | |
| | (Sorsby fundus dystrophy, | |
| | pseudoinflammatory) | |
| TNF | tumor necrosis factor (TNF | Osteoclast |
| | superfamily, member 2) | activity |
| TNFRSF10B | tumor necrosis factor receptor | Inflammation |
| | superfamily, member 10b | in bone |
| TNFRSF11B/ | tumor necrosis factor receptor | Osteoclast |
| OPG | superfamily, member 11b | activity |
| | (osteoprotegerin) | |
| TNFSF11/ | tumor necrosis factor (ligand) | Osteoclast |
| RANKL | superfamily, member 11 | activity |
| UNK_D83402 | prostaglandin I2 (prostacyclin) | HBM Bone |
| | synthase | |
| VCAM1 | Vascular cell adhesion molecule | Inflammation |
| | 1 | in bone |
| WISP2 | Wnt1 inducible signaling | HBM Bone |
| | pathway protein 2 | |
| WNT10B | wingless-type MMTV integration | Bone and |
| | site family, member 10B | colon cancer |
| WNT6 | wingless-type MMTV integration | HBM Bone |
| | site family, member 6 | |

The materials and methods relating to the protein and nucleic acid arrays for bone load are discussed in greater detail in International PCT Application No. PCT/US2004/17951, which is herein incorporated in its entirety for all purposes.

4.2 Functional Evaluation of Norrin in Xenopus

Xenopus embryos are an informative and well-established in vivo assay system to evaluate the modulation of Wnt signaling see, e.g., McMahon et al., 1989 Cell 58: 1075-84; Smith and Harland 1991 Cell 67: 753-65, reviewed in Wodarz and Nusse, 1998 Annu. Rev. Cell. Dev. Biol. 14: 59-88).

Modification of the Wnt signaling pathway by impacting the Norrin-Frizzled4-LRP5 complex can be visualized by examining the embryos for a dorsalization phenotype (duplicated body axis) after RNA injection into the ventral blastomere at the 4- or 8-cell stage. On the molecular level, phenotypes can be analyzed by looking for expression of various marker genes in stage 10.5 day embryos. Such markers would include general endoderm, mesoderm, and ectoderm markers as well as a variety of tissue-specific transcripts.

Analysis of the embryos can be done using RT-PCR/TaqMan® and can be done on whole embryo tissue or in a more restricted fashion (microdissection). Because this system is very flexible and rapid, by injecting combinations of transcripts, such as Norrin, LRP5/LRP6 and Fz4, the mechanism of Norrin signaling pathway can be dissected. Previous studies have demonstrated that LRP6 alone or in combination with LRP5+Wnt5a were able to induce axis duplication (dorsalization) in this system (Tamai et al., 2000 Nature 407: 530-35). Once the Norrin signaling is established, it can be used to evaluate by Dkk and Kremen antagonists and Norrin agonists and Norrin mimetics.

4.2.1 Constructs for Xenopus Expression (Vector pCS2⁺)

Norrin, LRP5/6, Fz4, Dkk (e.g., Dkk1), Wnt, and Kremen1/2 cDNAs can be subcloned into a vector, such as pCS2⁺, in the sense orientation with respect to the vector SP6 promoter. The pCS2⁺ vector contains an SV40 virus polyadenylation signal and T3 promoter sequence (for generation of antisense mRNA) downstream of the insert. Other vectors can also be utilized for expression of the proteins, in any combination.

4.2.2 mRNA Synthesis and Microinjection Protocol

mRNA for microinjection into Xenopus embryos can be generated by in vitro transcription using the cDNA constructs in the pCS2⁺ vector for example as described above as template. RNA is synthesized using the Ambion mMessage mMachine high yield capped RNA transcription kit (Ambion Cat. #1340) following the manufacturer's specifications for the Sp6 polymerase reactions. RNA products can be brought up to a final volume of 50 μL in sterile, glass-distilled water and purified over Quick Spin Columns for Radiolabeled RNA Purification using a G50-Sephadex column (Roche Cat. #1274015) following the manufacturer's specifications. The resulting eluate was finally extracted with phenol:chloroform:isoamyl alcohol and isopropanol precipitated using standard protocols (Sambrook et al., 1989). Final RNA volumes are usually approximately 50 μL. RNA concentration can be determined by absorbance values at 260 nm and 280 nm. RNA integrity can be visualized by ethidium bromide staining of denaturing (formaldehyde) agarose gel electrophoresis (Sambrook et al., 1989). Various amounts of RNA (about 2 pg to about 1 ng) are injected into the ventral blastomere of the 4- or 8-cell Xenopus embryo. These protocols are described in Moon et al., 1989 Technique-J. Meth. Cell & Mol. Biol. 1: 76-89, and Peng, 1991 Meth. Cell. Biol. 36: 657-62.

Molecules identified as modulating Norrin function or which act as Norrin mimetics in any of the assays described herein can be further validated using animal models or other in vitro screening assays.

4.3 Evaluation of Norrin for Osteogenic Effect in Mesenchymal Stem Cells

Human mesenchymal stem cells (hMSCs) (Cambrex Bio Science, Walkersville, Md.) and mouse stem cells (e.g., C3H10T1/2, ATCC) can be induced to differentiate into mineralized bone nodules (Jaiswal et al., 1997 J. Cell Biol. 64: 295-312) or adipose: tissues (Pettinger et al., 1999 Science 284: 143-147) in vitro by osteogenic or adipogenic medium respectively. Wnt signal activation enhances osteogenesis and inhibits adipogenesis in hMSCs. Norrin-Fz4-LRP5 mediated signaling is expected to provide a similar type of differentiation patterns in hMSCs. Thus, identifying Norrin mimetics and Norrin agonists using hMSCs (or other MSC cell from another vertebrate) is a screening assay contemplated.

In addition to human mesenchymal stem cells, stem cells from other vertebrate animals can also be used. Mesenchymal stem cells are progenitor cells to various bone cells (e.g., osteoblasts) as well as to adipocytes (see, e.g., Bennett et al., 2005 Proc. Nat'l Acad. Sci. USA 102(9): 3324-3329). Alternatively, more differentiated cells can be substituted such as preosteoblasts, osteocytes, and mature osteoblasts. It should be noted that instances wherein the cell line is indicated to be a human derived cell line, analogous cells from another vertebrate animal can be substituted.

4.3.1 Evaluation of Osteogenic Activity by Norrin Over Expression

Briefly, Norrin can be added to hMSCs (passage 3-6) as a purified protein or as part of conditioned medium, or expressed by infecting hMSCs using viral vectors. Alternatively, Norrin, Norrin agonists, and Norrin mimetics can be added to the hMSCs along with the osteogenic medium (growth medium supplemented with 10 nM dexamethasone, 50 μg/mL L-ascorbic acid and 5 mM beta-glycerophosphate). After about 1 to about 3 weeks of incubation along with appropriate control medium at about 37° C., and by weekly replenishment of fresh medium with or without Norrin, the osteogenic activity can be measured by standard techniques. For example, the osteogenic activity can be measured by staining the cells for alkaline phosphatase (AlkPhos) protein expression, determining the enzymatic activity of AlkPhos, induction of AlkPhos or osteocalcin mRNAs and detection of mineralization by Alizerin Red or von-Kossa stains along with appropriate controls.

4.3.2 Evaluation of Modulation of Adipogenesis by Norrin Over Expression

Norrin, Norrin agonists, or Norrin mimetics can be added to hMSCs by expressing using viral or other types of vectors along with the adipogenic differentiation medium (i.e., growth medium containing 10 nM dexamethasone, 50 μg/mL L-ascorbic acid phosphate, 500 μM isobutylmethylxanthine and 60 μM indomethacin) for 1-3 weeks. The effect of Norrin, Norrin agonists, or Norrin mimetics on adipocyte modulation can be determined by the alteration of the expression of adipogenic marker genes (e.g., Adipsin) or by staining the cells with oil red O reagent.

Changes in expression due to the administration of test agents can be performed using DNA array technology. Such technology is already used to test for obesity and diabetes. Therefore, one aspect would be to screen test agents using DNA array technology developed for obesity. See, e.g., Nadler et al., 2000 Proc. Natl. Acad. Sci. 97(21): 11371-11376, and the genes indicated for lipid metabolism, secreted proteins, as well as the other genes with decreased or increased expression associated with obesity. The cells used in conjunction with the DNA array technology can be mesenchymal cells, adipocytes, or preadipocytes, or other cells discussed herein.

In vivo changes in lipid levels and adipogenesis can be measured by a variety of different tests. Blood and serum can be collected and analyzed for blood chemistry. Thus, Norrin mimetics or Norrin agonists can be screened for the effects in vivo. Likewise, Dkk inhibitors and/or Kremen inhibitors can be screened for their impact on Norrin (in the presence or absence of a Norrin agonist) or Norrin mimetic activity with the LRP5/LRP6/Frizzled4 complex.

4.3.3 Evaluation of Osteogenesis and Adipogenesis Modulation by Norrin Gene Knock Down

By using an osteogenic or an adipogenic medium, hMSCs will differentiate into osteogenic or adipogenic lineages. Small hairpin RNAs of Norrin, Frizzled4 or LRP5 can be used as a control to demonstrate the enhancing effects of Norrin and Frizzled4 on the pathway and to show the impact inhibition of these proteins has on adipogenesis and osteogenesis. For example, in presence of osteogenic medium and the infection of viral vector containing Norrin shRNA, Norrin gene transcription can be blocked and the differentiation of hMSCs into osteoblasts or the production of mineralized bone nodules that can be detected by various methods indicated above.

Gene knockdown in tissue culture and in vivo can be attained by sequence-specific DNA or RNA analogs that can block the activity of selected single-stranded genetic sequences. Examples of such approaches include antisense oligonucleotide technology and the introduction of a homologous double-stranded RNA (dsRNA) or short interfering RNA (siRNA), which is also called post-transcriptional gene silencing (PTGS) or RNA interference (RNAi). This can be achieved by introducing into the cell siRNAs specific to the given target gene mRNAs via shRNAs (short hairpin RNAs) using various viral gene-delivery vectors or by transfecting plasmid vectors. Methods of performing RNA interference and gene silencing are known as discussed for example in Meister and Tuschl, 2004 “Mechanisms of gene silencing by double-stranded RNA,” Nature 431: 343-349; Dorsett and Tuschl, 2005 “siRNAs: applications in functional genomics and potential as therapeutics,” Nat. Rev. RNA Interference Collection 40-51 and the references cited therein. Once the siRNA is introduced, the extent of target gene knockdown is measured by standard techniques including qRT-PCR, Northern Blots for RNAs or by Western blots for protein expression.

5. Kits for Testing Agents Which Modulate Norrin

Another aspect contemplates kits for testing agents which modulate Norrin activity, and preferably for agents, which through modulation of Norrin activity, modulate the Wnt pathway. These kits can be used to screen for Norrin mimetics and Norrin agonists, as well as other mimetics and agonists of the LRP5/Norrin/Frizzled4 complex.

Contemplated kits would include cells and nucleic acids encoding at least Norrin and Frizzled4. Preferably, there would be nucleic acids encoding LRP5, Dkk (any of the Dkks), HBM, and/or Kremen (Kremen 1 and 2), as well as Norrin and Frizzled4 and/or any combination thereof. LRP6 and Wnts can also be included. Nucleic acids encoding the above polypeptides would be operably linked to a vector. Vector only would also be preferably included for control purposes. The kits could be styled for either transient transfection use or for stably transfecting cells.

Alternatively the kits can contain purified proteins, of any of the above proteins for use in vitro assay systems, such as those described here. They can include substrates such as nitrocellulose, ELSA plates, or other suitable substrates.

The kits would preferably include an assay appropriate reporter system, whether alkaline phosphatase, on or more fluorescent proteins and the like.

In one aspect, the kit could come with frozen cell lines for use in screening. In another aspect, the kit could come with instructions listing appropriate, previously tested cells that are suitable for the in vitro assays described herein.

In another aspect, the kit could come with the various reporters, enzymes, and reagents necessary for detecting the reporter used to detect the modulation. For example, if using the TCF-luci and TK-renilla assay system, the kit could come with TCF-luci and TK-renilla luciferases and detection reagents. Kits could also come with transgenic animals, wherein a cDNA for Dkk, Norrin, LRP5, LRP6, HBM, Kremen, Wnt, and/or Frizzled4 has been introduced into an animal(s). Kits can include a series of such animals for elucidation of activity for a particular test reagent.

6. Cell Lines

Another aspect the preparation of cell lines which do not express Norrin and/or Frizzled4. These cells lines can then have transiently or stably expressed non-native forms of LRP5, LRP6, Frizzled, Norrin, Dkk, Kremen, Wnt, and Norrin in any of the combinations discussed herein. Therefore, the cell lines can be used to screen reagents that are Norrin mimetics or Norrin agonists. For example, a cell line which has non-native (non-endogenous) forms of LRP5 and Frizzled4 expressed and lacks Norrin, there would be no means by which to activate the Wnt pathway through the LRP5-Frizzled4-Norrin mechanism. However, with the introduction of a Norrin mimetic, the pathway would be activated. Such cell lines would be useful controls for identifying Norrin mimetics. The cells could then have additional non-endogenous transcripts of Dkk and/or Kremen introduced with screening examined for test agents that modulate Dkk and/or Kremen interaction with the Frizzled4-LRP5/6-Norrin complex. Using this process, Dkk antagonists and/or Kremen antagonists can be identified. Introduction of a non-endogenous Norrin in one of these Norrin-free lines can be used to assess Norrin agonist on the Norrin-LRP5-Frizzled4 interaction.

Stable and transient expression of the nucleic acids encoding any of the proteins or biologically active polypeptide fragments thereof can be accomplished by means known in the art. See, e.g., R. IAN FRESHNEY, CULTURE OF ANIMAL CELLS: A MANUAL OF BASIC TECHNIQUE (2000) and JOSEPH SAMBROOK AND DAVID W. RUSSELL, MOLECULAR CLONING: A LABORATORY MANUAL (3^{rd} ed. 2001).

Cells which do not have endogenous Norrin include, but are not limited to, kidney cells. Therefore, kidney cells provide a useful tool for screening Norrin mimetics. Alternatively, cell lines can be prepared that are knock downs where one or more of the genes encoding LRP5, LRP6, Norrin, a Wnt, a Dkk, a Kremen, and/or Frizzled4 are knocked out such that an endogenous polypeptide can no longer be synthesized. This procedure can be carried out on any cell, such as but not limited to adipocytes, preadipocytes, mesenchymal cells, various bone cells, and kidney cells.

It will be apparent to those skilled in the art that various modifications and variations can be made in the materials and methods described herein without departing from the spirit or scope of the invention. Thus, it is intended that the present invention cover the modifications and variations of this invention provided they come within the scope of the appended claims and their equivalents.

### EXAMPLES

### Example 1

### Norrin/Wnt-TCF Signal Assay

Norrin clone isolation. cDNA was cloned by standard PCR method from the IMAGE clones. Specifically, the Norrin open reading frame (ORF) sequence from the NCBI (NM_{—}000266) was used to search for available IMAGE clones. Clone #5179578 was identified as a predicted full length cDNA. The IMAGE clone was purchased from Open Biosystems (Huntsville, Ala.). The ORF was amplified by standard PCR techniques using the following primers: 5′-CATATGAATTCACCATGAGAAAACATGTACTAGCTGCATC-3′ (SEQ ID NO:1) (which brings an EcoRI site for cloning as well as a consensus Kozak immediately 5′ of the initiating ATG) and 5′-GATATGCGGCCGCTCTAGATCAGGAATTGCATTCCTCGCAGTG-3′ (SEQ ID NO:2) (which brings both an XbaI and NotI site following the stop codon). The resulting PCR product was digested with EcoRI and NotI and cloned into the EcoRI and NotI sites of pcDNA3.1 (Invitrogen). Positive isolates were identified by restriction digest and confirmed by DNA sequence analysis to match the published sequence (Accession No. NM_{—}000266).

Kremen clone isolation. The PCR amplified full length fragment was subcloned into pcDNA3.1 vector at EcoRI/BamHI restriction enzyme sites. The isolated sequence was then verified to match the published human Kremen2 sequence (Accession Nos. NM_{—}172229/AB086405.1, and NP_{—}757384.1). cDNA was isolated from human osteoblast-like U20S cell line total RNA. Total RNA from about 2.5×10⁶ U20S cells was purified using the RNeasy kit (Qiagen, Valencia, Calif.) following the protocol of the manufacturer. Kremen2 cDNA isolate was amplified following standard PCR methods. The PCR primers used were: 5′ primer: 5′-GGACGAATTCACCATGGGGACACAAGCCCTGCAG-3′ (SEQ ID NO:3) 3′ primer: 5′-CTCGCTCATCTCCGCTCTCTGAGGATCCCAGG-3′ (SEQ ID NO:4). PCR amplified full length fragment was subcloned into pcDNA3.1 vector at EcoRI/BamHI restriction enzyme sites and the entire sequence was verified to match the published human Kremen2 sequence (Accession Nos. NM_{—}172229AB086405.1, NP_{—}757384).

Dkk clone isolation. A human cDNA with GenBank accession number AF127563 was available in the public database. Using this sequence, PCR primers were designed to amplify the open reading frame with a consensus Kozak sequence immediately upstream of the initiating ATG. Oligos 117162 (5′-CAATAGTCGACGAATTCACCATGGCTCTGGGCGCAGCGG-3′; SEQ ID NO:5) and 117163 (5′-GTATTGCGGCCGCTCTAGATTAGTGTCTCTGACAAGTGTGAA-3′; SEQ ID NO:6) were used to screen a human uterus cDNA library by PCR. The resulting PCR product was purified, subcloned into pCRII-TOPO (Invitrogen Corp.), sequence verified, and digested with EcoRI/XhoI. This insert was subcloned into the pCS2⁺ vector at the EcoRI-XhoI sites.

A full length cDNA encoding human Dkk2 was isolated to investigate the specificity of the Zmax/LRP5/HBM interaction with the Dkk family of molecules. Dkk1 was identified in yeast as a potential binding partner of Zmax/LRP5/HBM. Dkk1 has also been shown in the literature to be an antagonist of the Wnt signaling pathway, while Dkk2 is not (Krupnik et al., 1999). The Dkk2 full length cDNA serves as a tool to discriminate the specificity and biological significance of Zmax/LRP5/HBM interactions with the Dkk family (e.g., Dkk1, Dkk2, Dkk3, Dkk4, Soggy, their homologs and variant, etc.). A human cDNA sequence for Dkk2 (GenBank Accession No. NM_{—}014421) was available in the public database. Using this sequence, PCR primers were designed to amplify the open reading frame with a consensus Kozak sequence immediately upstream of the initiating ATG. Oligos 51409 (5′-CTAACGGATCCACCATGGCCGCGTTGATGCGG-3′; SEQ ID NO:7) and (5′-GATTCGAATTCTCAAATTTTCTGACACACATGG-3′; SEQ ID NO: 8) were used to screen human embryo and brain cDNA libraries by PCR. The resulting PCR product was purified, subcloned into pCRII-TOPO, sequence verified, and digested with BamHI/EcoRI. This insert was subcloned into the pCS2⁺ vector at the BamHI-EcoRI sites. For additional discussion regarding Dkk1 and Dkk2 clones, see International PCT application PCT/US02/15982. Similar constructions for Dkk3 and Dkk4 can be prepared using the sequences as referred to herein.

LRP6 clones. Full length LRP6 was isolated from the pED6dpc4 vector by XAhoI-XhaI digestion. The full length cDNA was reassembled into the XhoI-XhaI sites of pCS2⁺. Insert orientation was confirmed by DNA sequencing.

LRP5 (Zmax1) and HBM. Insert cDNA was isolated from the full length cDNA retrovirus constructs (with optimized Kozak sequences) by BglII-EcoRI digestion and subcloned into the BamHI-EcoRI sites of the pCS2⁺ vector. For more details on the LRP5 and HBM constructs, see U.S. Pat. No. 6,770,461 and International PCT Application PCT/US01/16946 entitled “Regulating lipid levels via the Zmax1 or HBM gene.”

Wnt clones. The Wnt genes utilized in the experiments shown herein were obtained as follows. Ten different full length Wnt cDNAs were purchased from Upstate Biotechnology (Lake Placid, N.Y.) in the vector pUSEamp(+). The genes are: Wnt1 (Cat. No. 21-121), Wnt2 (Cat. No. 21-122), Wnt3 (Cat. No. 21-123), Wnt3a (Cat. No. 21-124), Wnt4 (Cat. No. 21-125), Wnt5a (Cat. No. 21-133), Wnt5b (Cat. No. 12-126), Wnt6 (Cat. No. 21-127), Wnt7a (Cat. No. 21-128) and, Wnt7b (Cat. No. 21-129). Inserts were released by XbaI digestion and subcloned into the XbaI site of the pCS105 vector for Xenopus expression. Orientation was confirmed by sequence analysis.

Full length Wnt11 cDNA was isolated from 1×10⁶ human osteoblast cells (HOBs, passage #13) by RT-PCR using GCRich Kit (Clontech, Mountain View, Calif.) and the following specific primers: (Forward): 5′-GGGAATTCGCGACGATGAGGGCGCGGCCGCA-3′ (SEQ ID NO:9) (includes EcoRI site) and (Reverse): 5′-GGGCGGCCGCAGGGCCTCACTTGCAGACATAGC-3′ (SEQ ID NO: 10) (includes NotI site). The RT-PCR generated DNA fragment was digested with EcoRI and NotI and inserted into the EcoRI and NotI created site of the pcDNA3.1 vector. The full length sequence was verified to match the published Wnt11 sequence (Accession No. Y12692).

Full length cDNAs for other Wnt genes can be obtained by standard PCR techniques from various human cDNA library sources using public sequence to design primers to amplify the open reading frame of the gene and facilitate subcloning into the pCS105 vector or pcDNA3.1 type mammalian vector or other suitable mammalian vector. Suitable primers for other Wnt genes are presented in the Table 1 below. “F” stands for “forward” primer and “R” for “reverse” primer.

TABLE 1

SEQ

| | |
|---|---|
| Oligo | ID |

| | | |
|---|---|---|
| No. | Sequence | NO |

Wnt 10b-Accession No. U81787

| | | | |
|---|---|---|---|
| 51304 | F | 5-CTAACGGATCCACCATGCTGGAGGAGCCCCG-3 | 11 |
| 51306 | R | 5-GATTCGAATTCTCACTTACACACATTCACCC-3 | 12 |

Wnt 10a-Accession No. AY009400

| | | | |
|---|---|---|---|
| 118978 | F | 5-CTAACGGATCCACCATGGGCAGCGCCCACCC-3 | 13 |
| 118977 | R | 5-GATTCGAATTCTCACTTGCAGACGCTGACC-3 | 14 |

Wnt16a-Accession No. AF169963

| | | | |
|---|---|---|---|
| 51294 | F | 5-CTAACGGATCCACCATGGAAAGGCACCCACCC-3 | 15 |
| 51296 | R | 5-GATTCGAATTCTTACTTGCAAGTGTGGACATC-3 | 16 |

Wnt16-Accession No. AF152584

| | | | |
|---|---|---|---|
| 51298 | F | 5-CTAACGGATCCACCATGGACAGGGCGGCGCTC-3 | 17 |
| 51296 | R | 5-GATTCGAATTCTTACTTGCAAGTGTGGACATC-3 | 18 |

Wnt8d-Accession No. AY009402)

| | | | |
|---|---|---|---|
| 51300 | F | 5-CTAACGGATCCACCATGGGGGAACCTGTTTATGC-3 | 19 |
| 51302 | R | 5-GATTCTCTAGACTAGATATAGACCCCAAACC-3 | 20 |

Wnt8b-Accession No. Y11094

| | | | |
|---|---|---|---|
| 118973 | F | 5-CTAACGGATCCACCATGTTTCTTTCAAAGCCTTCTGT | 21 |
| | | G-3 | |
| 118980 | R | 5-GATTCGAATTCTTAGGGTTTTCTCCCGGGTTTG-3 | 22 |

Wnt2b2-Accession No. AB045117

| | | | |
|---|---|---|---|
| 118979 | F | 5-CTAACGATTCAACATGCTGAGACCGGGTGGTG-3 | 23 |
| 118974 | R | 5-GATTCTCTAGATCAGGTCTGGTCCAGCCAC-3 | 24 |

Wnt2b1-Accession No. AB045116

| | | | |
|---|---|---|---|
| 118975 | F | 5-CTAACTCTAGAATGTTGGATGGCCTTGGAG-3 | 25 |
| 118976 | R | 5-GATTCTCTAGATCAGGTCTGGTCCAGCCAC-3 | 26 |

Reporter Assay. The TCF assay involves a 16x-TCF reporter (containing 16 copies of Wnt-beta-catenin signal responsive TCF element with basal TK-promoter) and Luciferase gene. The construct contains 16 copies of the TCF binding sites placed upstream of a minimal TK (thymidine kinase) promoter and the luciferase gene in pGL3 vector (Promega, Madison, Wis.). The sequence of the four TCF binding sites (see paired sequences below) was generated by oligonucleotide synthesis approach and contains the following sequence with NheI and XhoI restriction enzyme sites at the 5′ and 3′ ends respectively. The underlined domains indicate the TCF binding sites. Both strands are provided. When the two strands anneal, NheI (5′) and XhoI (3′) compatible restriction sites are introduced for further cloning and they contain the TCF binding sites (SEQ ID NOS: 27 and 28 respectively):

5′-CTAGCGAGAACAAAGGAGATTCAAAGGAGATCAAAGGAGATCAAAGG
ACTAGTTC-3′
3′-TCGAGAACTAGTCCTTTGATCTCCTTTGATCTCCTTTGAATCTCCTT
TGTTCTCG-5′

TK-renilla (Promega Corp., WI) as internal assay normalization control; and pcDNA vector-based constructs of Norrin, Wnt1, Wnt3a, Dkk1, and Kremen2 cDNAs as discussed above, and a Frizzled4 construct (Origene Tech. Inc.; Rockville, Md.) are also part of the assay. Other vectors capable of expressing different forms of vertebrate Norrin, Wnt1, Wnt3a, Dkk1, and Kremen2 can be substituted.

The clones individually containing these genes are co-transfected into Human Embryonic Kidney (HEK)-293A cells (ATCC, Manassas, Va.) or a human osteosarcoma derived bone/osteoblast-like cell line, U20S (ATCC). The cells were cultured in Dulbecco's Minimum Essential Media (DMEM)(Invitrogen) or RPMI media (Invitrogen) supplemented with 10% heat-inactivated fetal bovine serum (FBS), 1% glutamax (Invitrogen), and 1% penicillin/streptomycin (Invitrogen).

HEK-293A cells (50,000 cells per well) or U20S cells (25,000 cells per well) were plated in 96-well plates. After 24 hours of incubation after plating (approximately 80-90% confluent), the media was replaced with 100 μL of fresh serum-free OPTIM media (Gibco/BRL). Both cell types were transfected with the 16x-TCF(TK)-Firefly Luciferase (0.3 μg/well) and TK-Renilla-luciferase (0.06 μg/well) using Lipofectamine 2000 transfection reagent (Promega; Madison, W is.) according to manufacturer's instructions. Experiments were performed in duplicate.

The test cDNA constructs were transfected at different concentrations, as needed. About 0.0005 μg/well to 0.05 μg/well cDNA of each of the constructs were used. The transfection was performed using Lipofectamine™ 2000 (Invitrogen) according to manufacturer's instructions. The DNA mix and reagent was incubated for 30 min. 50 μL/well of the DNA-reagent mix was added to the 100 μL of OPTIM media. The cells then were incubated for four hours at 37° C. The transfection medium was replaced with fresh 150 μL of DMEM or RPMI media on the HEK 293A and U20S cells, respectively. After 20-24 hours of incubation at 37° C. in a CO₂ incubator, the media was removed. The transfected cell monolayers were lysed by adding 150 μL of 1× lysis buffer of Dual Luci Reagent (Promega Corp.).

After 10 minutes, 20 μL of the lysate was transferred into a new 96-well white-plate (Packard/Costar). Cell lysates were mixed with 100 μL/well of LARII buffer (Dual Luci Reagent) and the Relative Luciferase Units (RLUs) were measured using a Packard Topcount NXT™ luminescence counter (Meriden, Conn.). This was followed by the addition of 100 μL/well of “stop & glo” reagent (Dual Luci Reagent), and the internal assay control renilla luciferase was measured using the Packard Topcount NXT™ luminescence counter.

The ratio of TCF-firefly-luci to renilla was calculated and is presented as bar graphs in the FIGS. 1-4. The experiments were done in quadruplicate with standard deviations calculated for the degree of error shown.

FIG. 1 indicates that when Norrin cDNA was transfected into human U20S cells, it gave approximately a 2- to 3-fold induction (pink slant lined bar) of TCF signal in the absence of Wnt cDNA transfection. However, the HEK-293A transfected cells did not produce any significant TCF-signal activation. FIG. 1 also shows that the co-transfection of Norrin and LRP5, one of its co-receptors into HEK-293A cells, did not activate the TCF-signal (purple checkered bar).

Interestingly, when the vectors containing the genes for Norrin (Nr) or LRP5 (L5) were co-transfected into U20S cells, TCF-signal was enhanced. The materials and methods utilized are as described above. See FIG. 1, right hand side. When both Norrin and LRP5 were co-transfected in U20S cells, TCF signal was synergistically enhanced (right most bar, FIG. 1)—approximately 6-fold over vector-alone (control). Since Norrin requires the Frizzled4 (Fz4) receptor in addition to the LRP5/6 co-receptor, the data implies that U20S cells contain Fz4, while HEK-293A cells lack the Fz4. Without Fz4, Norrin cannot induce TCF-signal and hence the lack of response in the HEK-293A cells (left hand side of FIG. 1).

### Example 2

### Fz4 is Required For Norrin Signaling

In order to evaluate the Fz4-Norrin interaction, the Fz4 and LRP5 cDNAs were transfected into both cell types which were also transfected with Norrin. Transfections were performed as discussed above in Example 1. Detection of TCF was performed and constructs used are as discussed in Example 1. Data was obtained in quadruplicate with the statistical analysis seen being a calculation of the standard deviation.

The HEK-293A cells show that there is no TCF response for vector only (V), LRP5 only (L5), Fz4 only (F4), or LRP5 and Norrin (L5+Nr). The addition of Norrin and Fz4 (F4+Nr) yield approximately a 6-fold increase in TCF over vector or Fz4 alone. The data in FIG. 2 demonstrates that in HEK-293A cells, the functional Fz4 was the limiting factor for Norrin-TCF-signal activation. It also indicates that in HEK-293A cells, the Norrin-Fz4 interaction probably utilizes the endogenous LRP5/6 receptors of the cells. In addition, the co-transfection of LRP5 along with Fz4 and Norrin (L5+F4+Nr) in HEK-293 cells results in further enhancement of TCF-signal, up to 16-fold over LRP5-only (L5) and Norrin-only (Nr) transfected HEK293A cells. See left side of FIG. 2.

In contrast, the same tests were conducted with U20S cells. The U20S cells yielded significantly greater TCF activity by co-transfection of Fz4 and Norrin (F4+Nr) over vector alone (V), LRP5 alone (L5), Frizzled4 alone (F4), and U20S cells co-transfected with LRP5 and Norrin (L5+Nr). See FIG. 2. The response in U20S cells was further enhanced when Fz4, LRP5, and Norrin (F4+L5+Nr) were all co-transfected into the cells (about 2-fold to about 6-fold). Such data in U20S cells probably indicates the presence of endogenous Wnt-signal components including Fz4 and LRP5/6 receptors.

### Example 3

### Norrin induced LRP5-Fz4-TCF Signal Can Be Inhibited Synergistically by Dkk1 and Kremen2 in U20S Cells

U2OS cells were transfected or co-transfected with blank vector (V), LRP5 (L5), Frizzled4 (Fz4), Norrin (Nr), Kremen2 (Krm2), or Dkk1 as indicated in FIG. 3 according to the assay procedures set forth in Example 1. Results were obtained in quadruplicate and the standard of deviation calculated therefrom.

FIG. 3 displays the ratio of TCF-luci to renilla signal modulation in U20S-TCF assays when transfected with the various cDNA constructs. On the right side of FIG. 3, the bars indicates that transfection of LRP5 (L5), Fz4 (Fz4), or Norrin (Nr), gave up to about 2- to 5-fold induction over vector control. However, co-transfection of both Fz4 and Norrin (Fz4+Nr) resulted in about a 15-fold induction of TCF signal.

The effect of Fz4 and Norrin was further enhanced by the expression of the LRP5 (L5) cDNA (i.e., tallest and darkest bar). The maximal activity of Fz4+Nr+L5 was inhibited partially by co-transfecting the cells with Dkk1. When both Dkk1 and Krm2 were added to cells co-transfected with LRP5, Fz4, and Norrin (L5+Fz4+Nr), the TCF-signal almost completely inhibited (right side, bar on far right).

The results displayed in FIG. 3 indicate that Norrin-LRP5-Fz4 mediated TCF-signal can be inhibited by Dkk1. Dkk1 is considered to be an efficient inhibitor of Wnt-LRP5-Fz induced Wnt-canonical pathway. Interestingly, Kremen2 (Krm2) addition synergizes with Dkk1 and in turn resulted in blocking the Norrin action of enhancing the Wnt pathway.

### Example 4

### Norrin Mediated TCF-signals with HBM and LRP5 are Differentially Inhibited by Dkk1 and Kremen2

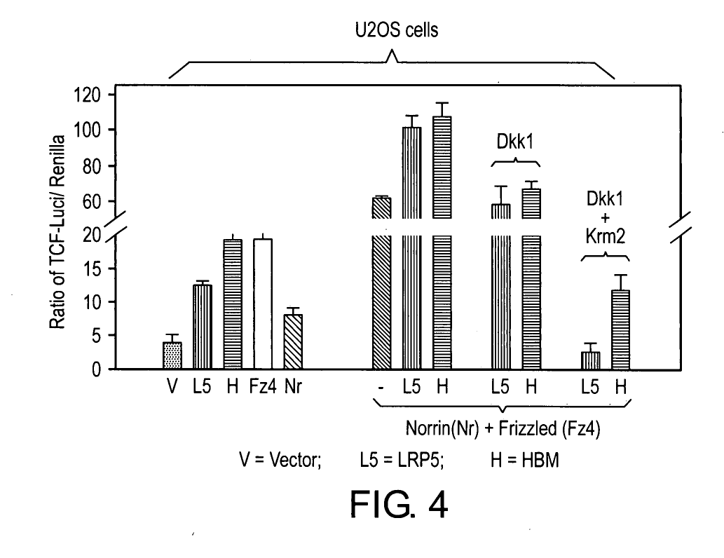

A comparison of Norrin-TCF-signal modulation by LRP5 or its gain of function mutant, HBM, was studied in cDNA transfected U20S cells. The results are displayed in FIG. 4. Transfection of U20S cells with HBM cDNA gave a slightly greater TCF-signal than transfection with LRP5 cDNA. See left side of FIG. 4, which shows vector only (V), LRP5-only (L5), HBM only (H), Frizzled4-only (Fz4), and Norrin (Nr). Constructions and conditions are as described for Example 1. The experiment was performed in quadruplicate with the standard of error calculated there from.

Co-transfection of the U20S cells with Norrin and Fz4, as well as either LRP5 (Nr+Fz4+L5) or HBM (Nr+Fz4+H), resulted in maximal TCF-signal with both the LRP5 and HBM cDNAs, i.e., about 25-fold over basal activity. Dkk1 co-transfection with the Norrin, Frizzled4, LRP5, or Norrin, Frizzled4, and HBM combinations, resulted in about a 38-40% inhibition of TCF signal. The Dkk1 inhibition was further enhanced with the addition of Kremen2 (Krm2). See right hand side of FIG. 2, two-right-most bars.

The comparative Norrin-TCF-signal analysis implies that LRP5 mutation G171 V mediated Norrin-Fz4-TCF signal confers a partial resistance to the inhibitory action of Kremen2 and Dkk1. This interesting observation is quite similar to the results previously observed with Wnt3a and Wnt1 mediated TCF-signal with LRP5 and HBM in presence of Dkk1 and Kremen2. It has been reported that LRP5-Wnt-TCF signaling modulates osteogenesis, while the HBM mutation leads to high bone mass phenotype in humans and in transgenic mice. Based on these results, it is likely that Norrin, as a more specific ligand of LRP5-Fz4 complex than Wnts, plays a significant role in bone metabolism.

In each of the examples above, Dkk1 can be substituted with Dkk2, Dkk3, and/or Dkk4. Additionally, in examples using Kremen2, it would be understood that Kremen1 could be substituted and used in the same assay. Additionally, the proteins or biologically active polypeptides can be introduced by co-transfections or by the addition of the purified protein and/or conditioned media containing the protein and/or biologically active polypeptides.

With the in vitro data discussed supra, Norrin has been shown to enhance the LRP5-Frizzled4 mediated Wnt-canonical pathway by activating TCF-reporter in U2OS bone cells and not in HEK-293A cells without the transfection of Frizzled4. LRP5-mediated Wnt signaling is important in bone formation/maintenance as evidenced by high bone mass (“HBM”) phenotype in LRP5-G171V mutation in humans and in transgenic animals. The data presented also shows that Norrin mediated TCF-signal in presence of LRP5-G171V (HBM) mutant is less sensitive to Dkk1 mediated inhibition as compared to that with LRP5. Since it is hypothesized that decreased inhibition due to G171V mutation in LRP5 is one of the causes of HBM phenotype, we would expect that Norrin, its expression, its induction, and/or Norrin mimetics could enhance bone formation or maintenance in vivo. Thus, a Norrin knockout in vivo could show osteopenia. The above assays are representative assays for use in screening, inter alia, Norrin mimetics, Norrin agonists, and Frizzled4 agonists.

All references cited herein are incorporated by reference herein in their entirety for all purposes.

What is claimed is:

1. A method of screening an agent that modulates bone metabolism or lipid metabolism comprising:
(a) having a Norrin protein or a biologically active Norrin polypeptide fragment and a Frizzled4 protein or a biologically active polypeptide fragment of Frizzled4 fused to LRP5 and/or LRP6 proteins or biologically active polypeptide fragments of LRP5 and/or LRP6 in the presence of the agent; and
(b) measuring at least one parameter of bone modulation and/or lipid modulation to screen for the agent that modulates bone metabolism or lipid metabolism.

2. The method of claim 1, wherein the agent is a Norrin mimetic.

3. The method of claim 1, wherein the parameter of bone metabolism modulation is of bone density, bone strength, trabecular number, bone size, or tissue connectivity, or any combination thereof.

4. The method of claim 1, wherein the parameter of lipid metabolism modulation is a change in the level of HDL, VLDL, cholesterol, triglyceride, apoE, or LDL.

5. The method of claim 1, wherein the bone metabolism parameter measured is altered expression of one or more of COX-2, Jun, Fos, cyclin D1, Wnt10B, SFRP1, connexin 43, eNOS, Wnt10B, cyclin D1, Frizzled2, and WISP2 is modulated.

6. The method of claim 5, wherein the bone metabolism parameter measured is altered expression of one or more of COX-2, Jun, Fos, cyclin D1, Wnt10B, SFRP1, connexin 43, and eNOS is modulated.

7. The method of claim 1, further comprising a Dkk protein or a biologically active Dkk polypeptide fragment.

8. The method of claim 1, further comprising a Kremen protein or a biologically active Kremen polypeptide fragment.

9. The method of claim 8, further comprising a Dkk protein or a biologically active Dkk polypeptide fragment.

10. The method of claim 1, further comprising a Wnt protein or a biologically active Wnt polypeptide fragment.

11. A method of screening an agent that modulates a Norrin-Frizzled4 activity comprising:
(a) having the agent, a Norrin protein or biologically active polypeptide fragment of Norrin, and a Frizzled4 protein or biologically active polypeptide fragment of Frizzled4 fused to LRP5 and/or LRP6 or biologically active polypeptide fragment of LRP5 and/or LRP6, or fused to a ligand binding domain (LBD) containing polypeptide fragment of LRP5, and:
(i) a Kremen protein; and/or
(ii) a Dkk protein; and
(b) determining whether the agent modulates a Norrin-Frizzled4 activity.

12. The method of claim 11, wherein the agent is a Norrin mimetic, Dkk antagonist, or a Kremen antagonist.

13. A method of identifying an agent that regulates bone modulation or lipid modulation comprising:
(a) administering the agent to a cell expressing Frizzled4 and LRP5, wherein Frizzled4 is a Frizzled4 protein or a biologically active Frizzled4 polypeptide, and LRP5 is a LRP5 protein or a biologically active polypeptide of LRP5;
(b) determining whether said administration of the agent modulates a LRP5-Frizzled4 interaction; and
(c) determining whether the agent modulates a bone parameter or a lipid parameter.

14. The method of claim 13, wherein the agent is a Norrin mimetic, a Dkk antagonist, or a Kremen antagonist.

15. The method of claim 13, wherein the cell does not express Norrin.

16. A method of screening an agent that regulates bone modulation or lipid modulation comprising:
(a) administering the agent to a cell expressing LRP5, Norrin and Frizzled4, wherein LRP5 is a LRP5 protein or a biologically active LRP5 polypeptide, Norrin is a Norrin protein or a biologically active Norrin polypeptide, and Frizzled4 is a Frizzled4 protein or a biologically active polypeptide of Frizzled4;
(b) determining whether said administration of the agent modulates Norrin-Frizzled4 interaction; and
(c) determining whether the agent modulates a parameter of bone modulation or lipid modulation.

17. The method of claim 16, wherein the cell expresses a non-endogenous Norrin, LRP5, and/or Frizzled4.

18. The method of claim 16, wherein the cell does not express an endogenous Norrin.

19. The method of claim 16, wherein LRP5 is co-expressed as a fusion polypeptide with Frizzled4.

20. The method of claim 16, wherein the cells are bone cells, kidney cells, mesenchymal cells, adipocytes, preadipocytes, or Xenopus cells.

21. The method of claim 16, wherein another series of cells co-expresses Norrin-Frizzled4, and Dkk, and determining whether said agent modulates Dkk inhibition of Norrin-Frizzled4.

22. The method of claim 16, wherein another series of cells co-expresses Norrin, Frizzled4, and Kremen, and determining whether said agent modulates Kremen inhibition of Norrin-Frizzled4.

23. The method of claim 21, wherein the Dkk is Dkk1, Dkk2, Dkk3, or Dkk4, or a biologically active polypeptide of Dkk1, Dkk2, Dkk3, or Dkk4.

24. The method of claim 22, wherein Kremen is Kremen1 or Kremen2, or a biologically active polypeptide of Kremen1 or Kremen2.

25. The method of claim 8, wherein the Kremen is Kremen1 or Kremen2 or a biologically active polypeptide of Kremen1 or Kremen2.

26. The method of claim 16, wherein the cells are bone cells, adipocytes, preadipocytes, stem cells, or kidney cells.

27. The method of claim 16, further comprising the step of administering the agent to an animal, and determining whether said agent induces a change in bone mass in said animal.

28. The method of claim 10, wherein Wnt is Wnt 1 to Wnt 19.

29. A cell or a cell line lacking a native Norrin and which expresses a non-native LRP5 and a non-native Frizzled4, wherein the non-native LRP5 is a non-native: LRP5 protein or a biologically active fragment thereof and the non-native Frizzled4 is a non-native Frizzled4 protein or a biologically active fragment thereof.

30. The cell line of claim 29, wherein the non-native LRP5 and/or the non-native Frizzled4 are stably expressed.

31. The cell line of claim 29, which further expresses a non-native Dkk and/or a non-native Kremen or biologically active polypeptide fragments of a non-native Dkk or non-native Kremen.

32. The cell line of claim 29, wherein the non-native Dkk is Dkk1, Dkk2, Dkk3, or Dkk4 and the non-native Kremen is Kremen1 or Kremen2.
